(19) United States
(12) Patent Application Publication
(10) Pub. No.: US 2009/0305995 A1
(43) Pub. Date: Dec. 10, 2009

(54) AGONISTS AND ANTAGONISTS OF THE SOMATOSTATIN RECEPTOR

(75) Inventors: Karl Heinz Krawinkler, Basel (CH); Peter Meier, Allschwill (CH); Bernard Faller, Obermorschwiller (FR)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG, Basel (CH)

(21) Appl. No.: 11/719,474

(22) PCT Filed: Nov. 14, 2005
(86) PCT No.: PCT/EP2005/012178

§ 371 Date: May 16, 2007

(30) Foreign Application Priority Data
Nov. 16, 2004 (GB) 0425258.1

(51) Int. Cl. A61K 38/07; C07D 209/16; A61K 31/40; C07K 5/10

(52) U.S. Cl. 514/18; 548/506; 514/419; 530/330

(57) ABSTRACT

The invention relates to substituted F3-Phe-trp-F 3-Lys-beta-tri-peptides and derivatives thereof, a process for their preparation, pharmaceutical preparations which contain these compounds which are agonists/antagonists of somatostatin receptors, as active agents for the treatment of disorders which can be influenced by a modulation of somatostatin receptor activity, in particular somatostatin receptor sst4 activity, by the compounds of the invention.

The invention relates to substituted β³-Phe-Trp-β³-Lys-beta-tri-peptides and derivatives thereof, a process for their preparation, pharmaceutical preparations which contain these compounds which are agonists/antagonists of somatostatin receptors, as active agents for the treatment of disorders which can be influenced by a modulation of somatostatin receptor activity, in particular somatostatin receptor sst₄ activity, by the compounds of the invention.

Somatostatin (SRIF) is a hormone which acts with G protein-coupled receptors to influence a variety of cellular processes. It naturally occurs in two major cyclic forms: as a tetradecapeptide and as a 28-amino acid form. It is known to affect cell growth and to inhibit the secretion of hormones and neurotransmitters such as catecholamine, insulin, growth hormone, Ghrelin, glucagon, gastrin, secretin and bile, among others. These diverse biological activities of SRIF are mediated by a family of five different receptors sst₁ to sst₅, which SRIF binds equally strongly in the low picomolar range. However, the extent of functional redundancy between the different somatostatin receptors is not known.

Somatostatin is currently thought to play a major role in the regulation of hormone/transmitter release, both in the brain and periphery, including gut, pancreas and lung. As a result, this peptide has pleiotropic effects on whole body/systemic functions, such as growth and homeostasis, where it influences the secretion of various mediators. In the brain, for example, somatostatin regulates the hypothalamic-pituitary axis, blocking the release of growth hormone.

The following details about the molecular mechanisms by which somatostatin controls secretion are known: somatostatin is a ligand for a family of 7TM G-protein-coupled receptors, sst₁ to sst₅, which differ in the distribution and the pathways to which they couple. Through G-proteins, these receptors affect several pathways, including inhibiting adenylate cyclase (AC) and cAMP signaling, and activating protein tyrosine phosphatases, PLD and PLA. These receptors also influence K, Ca and Na channel function and intracellular Ca mobilisation. These mechanisms enable the inhibition of hormone secretion and effects on proliferation by somatostatin. Specifically, via G-proteins sst₄ is known to inhibit cAMP signaling, active PLD and PLA2, alter Ca/H channel activity, inhibit Na/K exchanger NHI1 and activate the MAPK pathway. These pathways lead to an inhibition of exocytosis of synaptic residues and granules, including of GABA and glutamate release, and the promotion of proliferation.

Considering the pleiotropic effects of somatostatin, it is desirable to be able to selectively induce specific effects, in specific tissues if possible. While SRIF receptor subtypes have been characterized by molecular cloning and pharmacology, the availability of selective ligands for individual subtypes is still relatively limited. The first synthetic peptide analogues of SRIF, e.g. octreotide, bind with a similar affinity to two or more receptor subtypes. Recently however, Rivier et al. (2003) have developed octapeptides with a high selective affinity to the sst₄ receptor. Some of these peptides have proved to be clinically useful and are indicated for the treatment of acgromegaly, pancreatic tumors and other functional gastro-intestinal disorders, for example. Most of these peptide somatostatin agonists are rather unstable in vivo due to protease degradation. Furthermore, the few side effects of sst agonists so far reported include gastro-intestinal disorders, and the occurrence of cholesterol gall stones.

Sst₄ expression in rat (similar to human) occurs in the brain, gut and pancreas. It is also the sole somatostatin receptor expressed in the lung. In the brain, moderate but widespread expression is found in the cortex, where sst₄ colocalises with sst₂ on somatodendrites, in the hippocampus, where localization is different to and separate from sst₂ and is found in the hypothalamus and the pituitary. The specific role played by sst₄ in each of these organs is not known and is complicated by the presence of other ssts.

More recently, a series of non-peptide agonists, which are subtype selective and have a high receptor affinity, have been reported for each of the 5 human SRIF receptor subtypes (for a review see Weckbecker et al. 2003). When synthesising SRIF analogues, preservation of the core residues D-Trp⁸-Lys⁹ of SRIF has been thought to be an absolute prerequisite for full receptor recognition and bioactivity. Studies recently carried out by Grace et al. (2003) indicate that the backbone conformation of the peptide is not important in binding to the sst₄ receptor, but forms a scaffold to orient the side chain of the essentially important residues, namely indol at position 8, amino alkyl function at position 9 and an aromatic ring in the respective positions for effective receptor ligand binding.

Liu et al (1998) describe a non-peptide somatostatin derivative, NNC 26-9100, which utilizes a novel thiourea scaffold to mimic the Trp⁸ residue, a non-hetero aromatic nucleus to mimic Phe⁷ and a primary amine or other basic probe to mimic the Lys⁹ residue of somatostatin, resulting in an affinity of K_{D}=6 nM. Studies are currently in progress to evaluate the therapeutic potential for the treatment of glaucoma.

Souers et al. (2000) describe a subtype selective somatostatin mimetic prepared by incorporating conformational constraints into a nine membered heterocyclic scaffold having an affinity for the sst₄ receptor up to K_{D}=41 nM.

Using a glucose-based peptido-mimetic approach Hirschmann et al. (2003) obtained somatostatin analogues with a binding affinity of K_{D}=53 nM and enhanced water solubility.

By molecular modelling of the somatostatin pharmacore, Rohrer et al. (1998) isolated an sst₄ receptor selective compound from a combinatorial library. In binding and functional assays, L-803, 087 proved to be a hsst₄ receptor agonist (K_{D}=0.7 nM). L-803, 087 did not inhibit the secretion of growth hormone, insulin or glucagon.

Biomolecules (like peptides, nucleotides or steroids) are tolerated in the body and often show high affinities for biological target classes, but do often not fulfill criteria of oral bioavailability. In that sense, they are expected to have only low absorption and permeability, and are unattractive as candidates for drug development. Additionally, the fast proteolytic degradation of peptides based on α-amino acids resulting in a very short in vivo half life time is also a major drawback in the action of native somatostatin.

In order to overcome these problems, analogues of biomolecules, e.g. β-peptides having high affinity and selectivity for hsst4 receptors have been developed (Seebach et al., 2001, Gademann et al., 2001). These β-peptides, however, have only moderate oral bioavailability.

Thus, an object of the present invention was the provision of novel sst₄ receptor binding compounds with increased bioavailability, particularly for oral administration. Surprisingly, it was found that fatty acid conjugates of mixed α/β³-tetrapeptide-based somatostatin analogues have a higher affinity for the sst₄ receptor and improved pharmacologic properties, e.g. an improved bioavailability compared to known sst₄ receptor agonists. The compounds of the invention have emerged as a promising new class of somatostatin agonists by combining hsst4-receptor subtype selectivity with the resistance against proteolysis.

The invention relates to compounds of the general Formula I

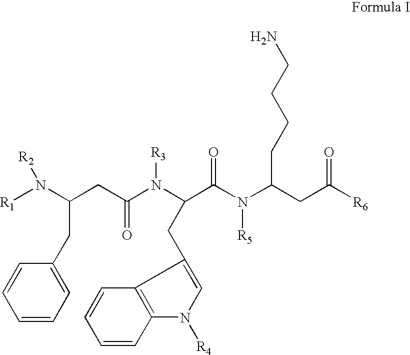

wherein R¹=COR⁷ or R⁷, wherein R⁷ is
a linear or branched C₁-C₁₂ alkyl group,
a linear or branched C₂-C₁₂ alkenyl group,
a linear or branched C₂-C₁₂ alkynyl group, or
a saturated/unsaturated, aromatic or heteroaromatic mono- or polycyclic group,
wherein said alkyl, alkenyl or alkynyl group may be mono- or polysubstituted with halo, hydroxy, C₁-C₄ alkoxy, carboxy, C₁-C₄ alkoxy carbonyl, amino, C₁-C₄ alkyl amino, di-(C₁-C₄-alkyl) amino, cyano, carboxy amide, carboxy-(C₁-C₄-alkyl) amino, carboxy-di(C₁-C₄-alkyl) amino, sulfo, sulfido (C₁-C₄-alkyl), sulfoxido (C₁-C₄-alkyl), sulfono (C₁-C₄-alkyl), thio or a saturated, unsaturated, aromatic or heteroaromatic, mono- or polycyclic group,
wherein said cyclic group may be mono- or polysubstituted with halo, hydroxy, C₁-C₄-alkoxy, carboxy C₁-C₄ alkoxycarbonyl, amino, C₁-C₄-alkylamino, di(C₁-C₄-alkyl) amino, cyano, carboxy amide, carboxy (C₁-C₄-alkyl) amido, carboxy-di(C₁-C₄-alkyl) amido, sulfo, sulfido (C₁-C₄-alkyl), sulfoxido (C₁-C₄-alkyl), sulfono (C₁-C₄-alkyl), thio, C₁-C₄ alkyl, C₂-C₄ alkenyl or C₂-C₄ alkynyl;
R² is hydrogen or C₁-C₄ alkyl,
R³ is hydrogen or C₁-C₄ alkyl, which may be substituted with a saturated, unsaturated, aromatic or heteroaromatic, mono- or polycyclic group,
R⁴ is hydrogen or C₁-C₄ alkyl,
R⁵ is hydrogen or C₁-C₄ alkyl, and
R⁶=(Y)ₙ(—NR⁸R⁹)ₘ, wherein Y is the residue of an amino carboxylic acid, particularly of a β-aminocarboxyclic acid, wherein Y may form a cyclic group;
n=0 or 1,
m=0 or 1,
R⁸ and R⁹ are independently hydrogen,
a linear or branched C₁-C₁₂ alkyl group,
a linear or branched C₂-C₁₂ alkenyl group,
a linear or branched C₂-C₁₂ alkenyl group,
or a saturated, unsaturated, aromatic or heteroaromatic mono- or polycyclic group,
wherein said alkyl, alkenyl or alkynyl group may be mono- or polysubstituted with halo, hydroxy, C₁-C₄ alkoxy, carboxy, C₁-C₄ alkoxy carbonyl, amino, C₁-C₄ alkyl amino, di-(C₁-C₄-alkyl) amino, cyano, carboxy amide, carboxy-(C₁-C₄-alkyl) amino, carboxy-di(C₁-C₄-alkyl) amino, sulfo, sulfido (C₁-C₄-alkyl), sulfoxido (C₁-C₄-alkyl), sulfono (C₁-C₄-alkyl), thio or a saturated, unsaturated, aromatic or heteroaromatic, mono- or polycyclic group,
wherein said cyclic group may be mono- or polysubstituted with halo, hydroxy, C₁-C₄-alkoxy, carboxy C₁-C₄ alkoxycarbonyl, amino, C₁-C₄-alkylamino, di(C₁-C₄-alkyl) amino, cyano, carboxy amide, carboxy (C₁-C₄-alkyl) amido, carboxy-di(C₁-C₄-alkyl) amido, sulfo, sulfido (C₁-C₄-alkyl), sulfoxido (C₁-C₄-alkyl), sulfono (C₁-C₄-alkyl), thio, C₁-C₄ alkyl, C₂-C₄ alkenyl or C₂-C₄ alkynyl;
or wherein R⁸ and R⁹ together form a cyclic group, preferably a 5- or 6-membered cyclic group;
or salts or derivatives thereof in the form of individual enantiomers, diastereomers or mixtures thereof.

Preferred are compounds of Formula I in which R⁷ can be either an unsubstituted or a substituted C₁-C₁₀ alkyl residue or an unsubstituted or a substituted cyclic group. Particularly preferred are methyl, ethyl, butyl, nonyl, cyclohexyl, phenyl, ethylphenyl and adamantyl.

R² is preferably hydrogen or methyl. R³ is preferably hydrogen, methyl, phenyl or ethyl. Preferably, R⁴ and R⁵ are independently hydrogen and methyl residues. More preferably, R⁴ and R⁵ are hydrogen.

The substituent n may be 0 or 1. When n=1, Y is preferably a β-amino acid residue, wherein R⁸ is an unsubstituted or a substituted C₁-C₁₀, particularly C₂-C₈ alkyl group or an unsubstituted or a substituted cyclic group, e.g. a β-threonine residue which may form a lactone group or a β-valine residue or a β-amino acid derivative, particularly a β-amino acid amide, e.g. an optionally substituted β-threonine amide or β-valine amide.

The substituent m is preferably 1, i.e. is present, for example, as an amide group as indicated above. Preferably, at least one of R⁸ and R⁹ is an unsubstituted or a substituted C₁-C₁₀, particularly C₂-C₈ alkyl group or an unsubstituted or a substituted cyclic group.

R⁸ is more preferably ethyl, butyl, pentyl, hexyl, ethylphenyl or cyclopentyl. When R⁹ is other than hydrogen, it is preferably an unsubstituted C₁-C₂ alkyl group, e.g. methyl or ethyl.

Specific examples of the compounds of the present invention preferably include those compounds of Formula I in which R¹ represents COR⁷ and R⁶ represents a β-threonine amide. These are the compounds of Formula Ia according to the present invention

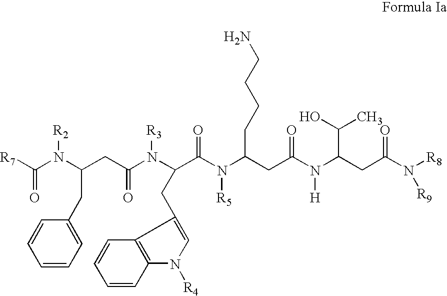

wherein R₇, R₂, R₃, R₄, R₅, R₈ and R₉ are as defined above.

Further preferred examples of the compounds of the present invention are those compounds of Formula I wherein R¹=COR⁷ and R⁶ represents threonine lactone. These are the compounds of Formula 1b according to the present invention

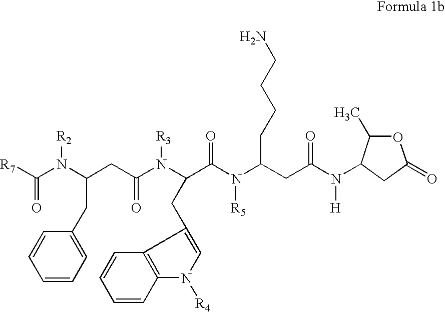

wherein R₇, R₂, R₃, R₄, R₅ are as defined above.

Preferred examples of the compounds of the present invention are those compounds of Formula I wherein R¹=COR⁷ and R⁶ represents a β-valine-amide. These are the compounds of Formula Ic according to the present invention

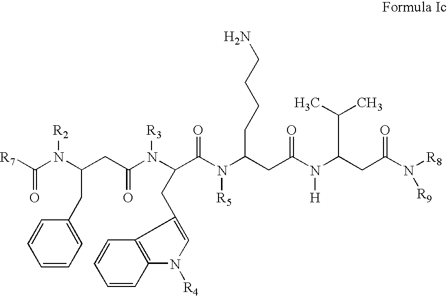

wherein R₇, R₂, R₃, R₄, R₅, R₈ and R₉ are defined as above.

Further preferred examples of the compounds of the present invention include those compounds of Formula I wherein R¹=COR⁷, and R⁶=NR⁸R⁹. These are the compounds of Formula 1d according to the present invention

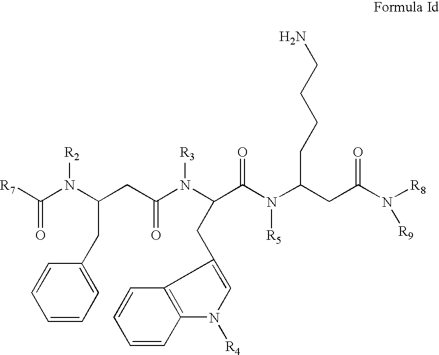

wherein R₇, R₂, R₃, R₄, R₅, R₈ and R₉ are as defined above.

The invention also relates to the physiologically acceptable salts and derivates of the compound of Formula I.

The physiologically acceptable salts may be obtained in a conventional way by neutralizing the acids with inorganic or organic bases. Examples of suitable inorganic acids are hydrochloric acid, sulfuric acid, phosphoric acid or hydrobromic acid, and examples of suitable organic acids are carboxylic acid or sulfonic acids, such as acetic acid, tartaric acid, lactic acid, propionic acid, glycolic acid, malonic acid, maleic acid, fumaric acid, tannic acid, succinic acid, alginic acid, benzoic acid, 2-phenoxybenzoic acid, 2-acetoxybenzoic acid, cinnamic acid, mandelic acid, citric acid, malic acid, salicylic acid, 3-aminosalicylic acid, ascorbic acid, embonic acid, nicotinic acid, isonicotinic acid, oxalic acid, amino acids, methanesulfonic acid, ethanesulfonic acid, 2-hydroxyethanesulfonic acid, ethane-1,2-disulfonic acid, benzenesulfonic acid, 4-methylbenzenesulfonic acid or naphthalene-2-sulfonic acid. Examples of suitable inorganic bases are sodium hydroxide solution, potassium hydroxide solution, ammonia and suitable organic bases are amines, but preferably tertiary amines such as trimethylamine, triethylamine, pyridine, N,N-dimethylaniline, quinoline, isoquinoline, α-picoline, β-picoline, γ-picoline, quinaldine or pyrimidine.

Physiologically acceptable salts of the compounds of Formula I can additionally be obtained by converting derivatives having tertiary amino groups in a manner known per se with quaternizing agents into the corresponding quaternary ammonium salts. Examples of suitable quaternizing agents are alkyl halides such as methyl iodide, ethyl bromide and n-propyl chloride, but also arylalkyl halides such as benzyl chloride or 2-phenylethyl bromide.

The invention also relates to derivatives of the compounds of Formula I which are preferably compounds which are converted, e.g. hydrolyzed, under physiological conditions to compounds of Formula I or into which the compounds of Formula I are metabolized under physiological conditions.

The invention further relates to optical enantiomers or diastereomers or mixtures of compounds of Formula I which contain an asymmetric carbon atom, and in the case of a plurality of asymmetric carbon atoms, also the diastereomeric forms. Compounds of Formula I which contain asymmetric carbon atoms and which usually result as racemates can be separated into the optically active isomers in a manner known per se, for example with an optically active acid. However, it is also possible to employ an optically active starting substance from the outset, in which case a corresponding optically active or diastereomeric compound is obtained as the final product.

The compounds of the invention have been found to have pharmacologically important properties which can be utilized in therapy. The compounds of Formula I can be employed alone, in combination with one another or in combination with other active ingredients.

The compounds of the present invention are β-peptide derivatives with a high affinity to human somatostatin receptors, particularly to the hsst4 receptor and high bioavailability. Preferably, the K_{D} is ≦ about 2 μM, more preferably the K_{D} is ≦200 nM and most preferably the K_{D} is ≦ 50 nM. Thus, an aspect of the invention that the compounds of Formula I or the salts thereof can be used for the treatment of disorders in which a modulation of hsst₄-signaling is beneficial. This modulation includes effects on the differentiated gene expression in response to the compounds of Formula I. This includes groups of genes related to the known molecular mechanism/signaling of sst₄ activity, such as calcium regulators, sodium calcium and potassium channels, MAP kinases, phosphatases and cAMP signaling. Via these mechanisms, sst₄ affects growth, metabolism, hormonal regulation and secretion of hormones. For instance, sst₄-signaling can affect proliferation via MAPK signaling, ERK, p53 and Rb and phosphatases (Patel, 1999; Weckbecker et al. 2003). The sst₄ receptor can also affect secretion via inhibition of cAMP/Ca²⁺-signals or via modulation of Ca/K channels on phosphotidylinositol signaling via phosphalipases. Linked to sst₄ activity are also genes for neurotransmitters/hormones such as VEGF (Mentelein et al., 2001) and glutamate (Moneta et al., 2002).

Examples of disorders and diseases which can be treated by sst4 receptor agonists such as the compounds of the invention are reported in WO2005082844, which teaching is incorporated herein by reference. Disorders arising from this sst₄ receptor activity include disorders of the central nervous system, in particular epilepsy, impaired behaviour such as impaired learning and memory or attention deficit disorder and pain, including chronic pain. Further possible uses are the treatment of patients suffering from neurological disorders, such as neurodegenerative diseases, in particular Alzheimer's disease, Parkinson's disease and multiple sclerosis.

The compounds of the invention can likewise be used for the treatment of hyperproliferative disorders, in particular of endocrine and solid tumors, for example for the treatment of acromegaly, melanomas, breast cancer, prostate adenomas and prostate cancer, lung cancer, bowel cancer, skin cancer and leukemias.

The compounds of the invention can be used for the treatment of diseases associated with vascular remodelling such as restenosis or the treatment of chronic transplant rejection. It can also be used for the treatment of post-surgical symptoms, such as brain aneurysms and postsurgical vascular re-stenosis. The compounds of the invention can be used for the treatment of wounds, the promotion of wound healing or tissue repair.

The compounds of the invention can be used for the treatment of gastrointestinal disorders such as diarrhoea and chemotherapy-induced and AIDS-related diarrhoea, as well as in the treatment of acute variceal bleeding. The compounds of the invention can be used for the treatment of inflammatory disorders including inflammations of the joints, including arthritis and rheumatoid arthritis, and other arthritic disorders such as rheumatoid spondylitis. Also possible is the treatment of psoriasis, Graves disease and inflammatory bowel disease.

Further possible use of the compounds of the invention are the treatment of allograft rejection. The compounds of the invention can be used for the treatment of diabetic retinopathy and nephropathy and diabetic angiopathies.

The compounds of the invention can be used in the treatment of ophthalmologic disorders, for example, age-related macula degeneration and glaucoma diabetic retinopathy. The compounds of the invention can also be used in the treatment of benign prostatic hyperplasia.

The compounds of the invention can also be labelled and used for diagnosis, e.g. radiodiagnosis and/or radiotherapy of SRIF receptor-expressing tumors, as well as the regression of otherwise unresponsive tumors.

The drug products are produced by using an effective dose of the compounds of the invention or salts thereof, in addition to conventional adjuvants, carriers and additives. The dosage of the active ingredients may vary depending on the route of administration, the age and weight of the patient, the nature and severity of the disorders to be treated and similar factors. The daily dose may be given as a single dose to be administered once a day, or divided into 2 or more daily doses, and is usually 0.001-100 mg. Daily dosages of 0.1-50 mg are particularly preferred.

Oral, parenteral, intravenous, transdermal, topical, inhalational and intranasal preparations are suitable as administration forms. Topical, inhalational and intranasal preparations of the compounds of the invention are particularly preferred. Galenical pharmaceutical presentations such as tablets, coated tablets, capsules, dispersible powders, granules, aqueous solutions, aqueous or oily suspensions, syrup, solutions or drops are used.

Solid drug forms may comprise inert ingredients and carriers such as, for example, calcium carbonate, calcium phosphate, sodium phosphate, lactose, starch, mannitol, alginates, gelatin, guar gum, magnesium stearate or aluminium stearate, methylcellulose, talc, colloidal silicas, silicone oil, high molecular weight fatty acids (such as stearic acid), agar-agar or vegetable or animal fats and oils, solid high molecular weight polymers (such as polyethylene glycol); preparations suitable for oral administration may, if desired, comprise additional flavourings and/or sweetners.

Liquid drug forms can be sterilized and/or, where appropriate, can comprise excipients such as preservatives, stabilizers, wetting agents, penetrants, emulsifiers, spreading agents, solubilizers, salts, sugars or sugar alcohols to control the osmotic pressure or for buffering and/or viscosity regulators.

Examples of such additions are tartrate buffer and citrate buffer, ethanol, complexing agents (such as ethylenediaminetetraacetic acid and its non-toxic salts). Suitable for controlling the viscosity are high molecular weight polymers such as, for example, liquid polyethylene oxide, microcrystalline celluloses, carboxymethylcelluloses, polyvinylpyrrolidones, dextrans or gelatin. Examples of solid carriers are starch, lactose, mannitol, methylcellulose, talc, colloidal silicas, higher molecular weight fatty acids (such as stearic acid), gelatin, agar-agar, calcium phosphate, magnesium stearate, animal and vegetable fats, solid high molecular weight polymers such as polyethylene glycol.

Oily suspensions for parenteral or topical uses may be vegetable, synthetic or semisynthetic oils such as, for example, liquid fatty acid esters with, in each case, 8 to 22 C atoms in the fatty acid chains, for example palmitic, lauric, tridecyclic, margaric, stearic, arachic, myristic, behenic, pentadecyclic, linoleic, elaidic, brasidic, erucic or oleic acid, which are esterified with monohydric to trihydric alcohols having 1 to 6 C atoms, such as, for example, methanol, ethanol, propanol, butanol, pentanol or isomers thereof, glycol or glycerol. Examples of such fatty acid esters are commercially available miglyols, isopropyl myristate, isopropyl palmitate, isopropyl stearate, PEG 6-capric acid, caprylic/capric esters of saturated fatty alcohols, polyoxyethylene glycerol trioleates, ethyl oleate, waxy fatty acid esters such as artificial duch preen gland fat, coco fatty acid, isopropyl ester, oleyl oleate, decyl oleate, ethyl lactate, dibutyl phthalate, diisopropyl adipate, polyol fatty acid esters inter alia. Also suitable are silicone oils differing in viscosity or fatty alcohols such as isotridecyl alcohol, 2-octyldodecanol, cetylstearyl alcohol or oleyl alcohol, fatty acids such as, for example, oleic acid. It is also possible to use vegetable oils such as caster oil, almond oil, olive oil, sesame oil, cottonseed oil, peanut oil or soybean oil.

Suitable solvents, gel formers and solubilizers are water or water-miscible solvents. Suitable examples are alcohols such as, for example, ethanol or isopropyl alcohol, benzyl alcohol, 2-octyldodecanol, polyethylene glycols, phthalates, adipates, propylene glycol, glycerol, di- or tripropylene glycol, waxes, methyl Cellosolve, Cellosolve, esters, morpholines, dioxane, dimethyl sulfoxide, dimethylformamide, tetrahydrofuran, cyclohexanine, etc.

Film formers which can be used are cellulose ethers able to dissolve or swell both in water and in organic solvents such as, for example, hydroxypropylmethylcellulose, methylcellulose, ethylcellulose or soluble starches.

Combined forms of gel formers and film formers are also possible. In particular, ionic macromolecules are used for this purpose, such as, for example, sodium carboxymethylcellulose, polyacrylic acid, polymethylacrylic acid and salts thereof, sodium amylopectin semiglycolate, alginic acid or propylene glycol alginate as sodium salt, gum arabic, xanthan gum, guar gum or carrageenan.

Further formulation aids which can be employed are glycerol, paraffin of differing viscosity, triethanolamine, collagen, allantoin, novantisolic acid.

It may also be necessary to use surfactants, emulsifiers or wetting agents for the formulation, such as, for example, Na lauryl sulfate, fatty alcohol ether sulfates, di-Na—N-lauryl-β-iminodipropionate, polyethoxylated castor oil or sorbitan monooelate, sorbitan monostearate, polysorbates (e.g. Tween), cetyl alcohol, lecithin, glyceryl monostearate, polyoxyethylene stearate, alkylphenol polyglycol ether, cetyltrimethylammonium chloride or mono/dialkylpolyglycol ether orthophosphoric acid monoethanolamine salts.

Stabilizers such as montmorillonites or colloidal silicas to stabilize emulsions or to prevent degradation of the active substances, such as antioxidants, for example tocopherols or butylated hydroxyanisole, or preservatives such as p-hydroxybenzoic esters, may likewise be necessary where appropriate to prepare the desired formulations.

Preparations for parenteral administration may be present in separate dose unit forms such as, for example, ampoules or vials. Solutions of the active ingredient are preferably used, preferably aqueous solutions and especially isotonic solutions, but also suspensions. These injection forms can be made available as a finished product or be prepared only immediately before use by mixing the active compound, e.g. the lyophilistate, where appropriate with further solid carriers, with the desired solvent or suspending agent.

Intranasal preparations may be in the form of aqueous or oily solutions or of aqueous or oily suspensions. They may also be in the form of lyophilistates which are prepared before use with the suitable solvent or suspending agent.

The manufacture, bottling and closure of the products takes place under the usual antimicrobial and aseptic conditions.

The invention further relates to a process for the manufacture of the compounds of the invention (FIG. 1).

According to the present invention, the compounds of general Formula I are manufactured according to the definitions for R¹, R², R³, R⁴, R⁵, R⁶, R⁷, R⁸ and R⁹ as previously given such that the synthetic protocol involves three efficient peptide coupling steps employing the same chemical reagents and three Boc-cleavage reactions using HCl in 1,4-dioxane. As the five-ring lactone demonstrates to be very stable against ring opening even when treated with strong carboxylic acid activating agents, the synthon can be used in all peptide coupling steps without utilization of protecting groups. With the growing peptide chain, solubility becomes a major concern. The final N-Boc-protected mixed α/β³-tetrapeptide proves to be potentially insoluble in lots of standard solvents used in peptide chemistry. The restricted, but partial solubility of the scaffold molecule in dichloromethane is sufficient to purify intermediate compounds by liquid/liquid extraction. Purification is finally achieved by extraction under weak acidic conditions established with aqueous citric acid, in order to prevent partitioning of the fully protonated product molecule (a weak base) between aqueous and organic phase.

After N-terminal-derivatization of the mixed (α/β³)-tetrapeptide scaffold with fatty acid analogues in parallel synthesis mode, deprotection of the Cbz-protecting group was achieved by hydrogenolysis (Pd on activated charcoal) in DMA under acidic conditions. Addition of trifluoroacetic acid to the solvent led to an acceleration of the hydrogenation process. In addition, immediate protonation of the so-generated primary amine inhibited a (possible) nucleophilic attack on the adjacent C-terminal five-ring-lactone. Thus, the formation of a macrocyclic lactam could be prevented. The final products were then purified by RP-chromatography leading to purities >95% as determined by HPLC, HR-MS, MS, LC-MS, 1D- and 2D-NMR spectroscopy.

The C-terminal five-ring lactones can be exchanged for their corresponding open-chain amide analogues. This was achieved by reacting the fatty acid derivatized-(α/β³)-tetrapeptides with ammonia in methanol. Due to the folding and unique structural properties of these β-amino acid containing tetrapeptides, initial reaction times range from 24 hours (nonanoyl-derivative, compounds 16 and 17 in Table 1) to 36 days (cyclohexyl-derivative, compound 26). Nonetheless, the reaction times can be accelerated by dissolving the lactone containing tetrapeptides in N,N-dimethylacetamide (DMA) and subsequent addition of ammonia in methanol. Conversion rates are generally near hundred percent (>98%) and due to the high purity (>95% as determined by RP-HPLC) of the generated C-terminal amides, further purification was not necessary.

In subsequent peptide series, primary or secondary amine building blocks are introduced into the peptide by reaction of the fully protected C-terminal β³-amino acids (N_{α}-Boc-N_{ω}-Z-(S)-β³-HLys and Boc-(R)-β³-Leucine) employing carbonyldiimidazole activation chemistry, followed by deprotection and subsequent coupling.

Double conjugated biomolecules (Formula Id, R⁶=NR⁸R⁹) consisting of only three amino acids (two β³ and one α) show much better solubility in organic solvents and lead to an acceleration in work up procedures by avoiding hardly separable emulsions. The same is observed for beta-peptides when capped with N-alkylated groups in the amide backbone.

### Bioactivity of the Compounds

The generated peptides are tested for their affinity to bind to human SRIF receptors expressed in Chinese hamster lung fibroblast (CCL39) cells. This is achieved in radioligand-binding assays, a displacement experiment in which the concentration of a substance is measured which is necessary for the replacement of 50% of a specifically bound radioligand ([¹²⁵I]LTT-SRIF₂₈). Specific binding is measured as the total binding of receptor-specific radioligand minus the amount of radioligand bound in presence of unmarked SRIF-14 (100 nM, nonspecific binding).

TABLE 1
List of all tested compounds based on scaffold I with the corresponding compound numbers.

| | N-terminal | | | | | | | | |
|---|---|---|---|---|---|---|---|---|---|
| | R1 | | | | | | | | |
| Building Blocks R2 C-terminal | 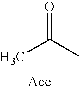 | 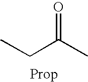 | 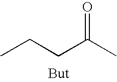 | 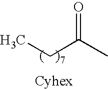 | 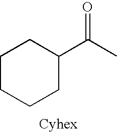 | 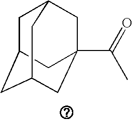 | 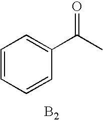 | 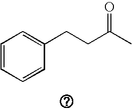 | H |
| 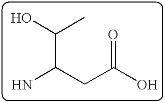 | 1, 2, 3 | | | 16, 17 | 26 | 18 | 14 | 15 | 35, 36 |
| 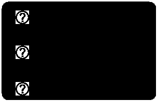 | 7, 8, 9 39 (R3 = Me) 21 (R4 = Me) | | | 11, 12 | 5 | 13 20 (R4 = Me) | 6 | 10 19 (R4 = Me) | 28, 29 38 (R3 = Me) |
| 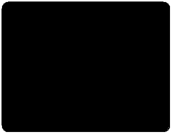 | 22 | | | | | | | 24 | |
| 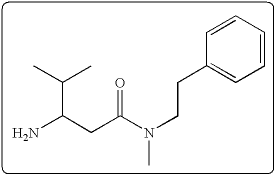 | 25 | | | | | 27 | | 23 | |
| 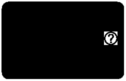 | 30, 31 | 46 (R3 = Me) 57 | 56 | | | 33, 34 | | 32 | 62 |
| 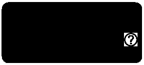 | 52 | 53 | 54 | | | | | | |
| 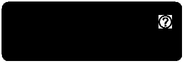 | 44, 45 | 50, 51 | 55 | | | | | | |
| 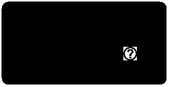 | | 43 | 42 | | | | | | |
| 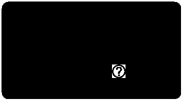 | 47 | 48, 49 | 41 | | | | | | |
| 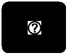 | 58 | 60 | 61 | | | | | | 59 |

Scaffold I:
R3 and R4 = H if not otherwise depicted

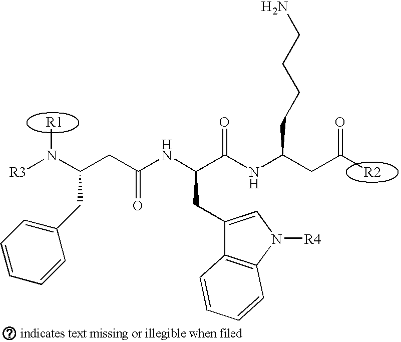

TABLE 2
List of all tested compounds based on scaffold II with the
corresponding compound numbers.

| | N-terminal | | |
|---|---|---|---|
| | R1 | | |
| Building Blocks R2 C-terminal | 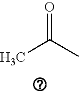 | 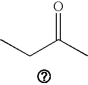 | H |
| 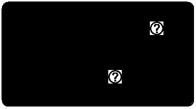 | | 63, 64 48, 67 (R5 = [symbol] | |
| 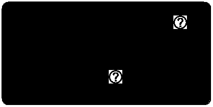 | | 65 68, 69 (R5 = [symbol] | |
| 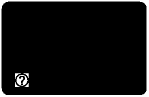 | 49 37 (R4 = Me) | | |
| 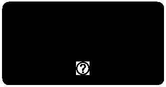 | | 70 | |
| 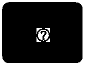 | | | 71, 72 |

Scaffold II:
R3 and R4 = H, R5 = Me if not otherwise depicted

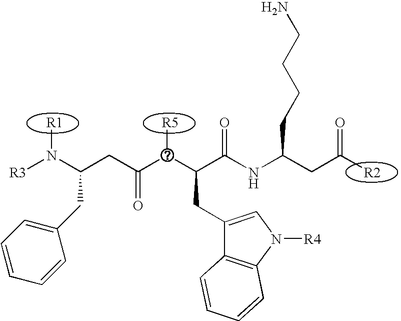

[symbol]

indicates data missing or illegible when filed

The compounds indicated in Tables 1 and 2 have moderate to high binding affinity and selectivity for the cloned hsst4 receptor. For the compound series row 1 and 2 in Table 1, activities given as respective K_{D}-values ranged from 60 nM (compounds 7-9) to 1202 nM (compound 5) for the more potent C-terminal (R)-4-amino-5-(R)-methyl-dihydro-furan-2-ones (β-homothreonine-lactone) molecules and from 170 nM (compounds 1-3) to 6166 nM (compound 18) for the C-terminal β-homothreonine-amide derivatives. As can be seen from FIG. 1, selectivities are changing within different compound series. A decrease in binding affinity leads consequently to a decline in receptor subtype selectivity. Highest binding affinity, however, is found for the hsst4 receptor in almost all cases.

### FIGURE LEGEND

FIG. 1 shows the binding data for the first compound series demonstrating the hsst4 selectivity of the peptide analogues.

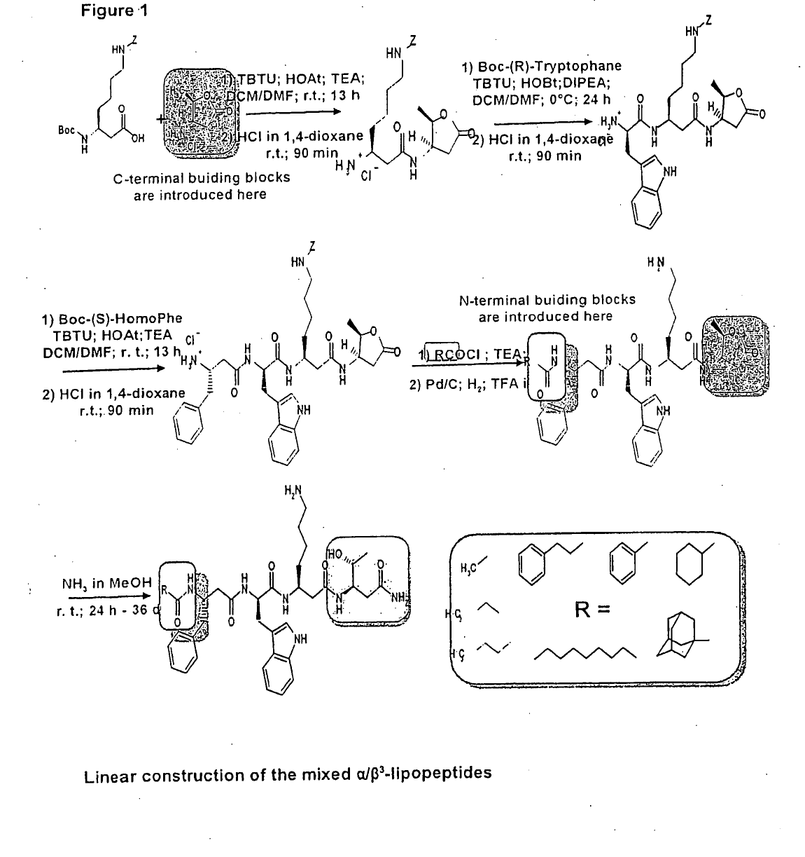

FIG. 2 shows the structure-activity relationships for the established double conjugated biomolecules. For positions R₁ and R₂ see scaffold I in Table I.

FIG. 3 shows biological screening for the best lipophilization positions.

FIG. 4 shows the binding affinities and selectivities for C-terminal modified compounds.

FIG. 5 shows the structure-activity relationships for the established double conjugated biomolecules.

FIG. 6 shows the binding affinities for C-terminal N-methylated compounds.

FIG. 7 shows the Correlation of RP-chromatographic retention times with ClogP values.

FIG. 8 shows the correlation of RP-chromatographic retention times with HT-LogP o/w values.

FIG. 9 shows correlation of HT-logP o/w with Clog P values.

FIG. 10 shows high throughput solubility data S_{w} measured at pH 6.8 (Wang-J et al, 2000, Linpinsky et al., 1997).

FIG. 11 shows high throughput permeability data log Pₑ (Pₑ in cm/s) measured at pH 6.8.

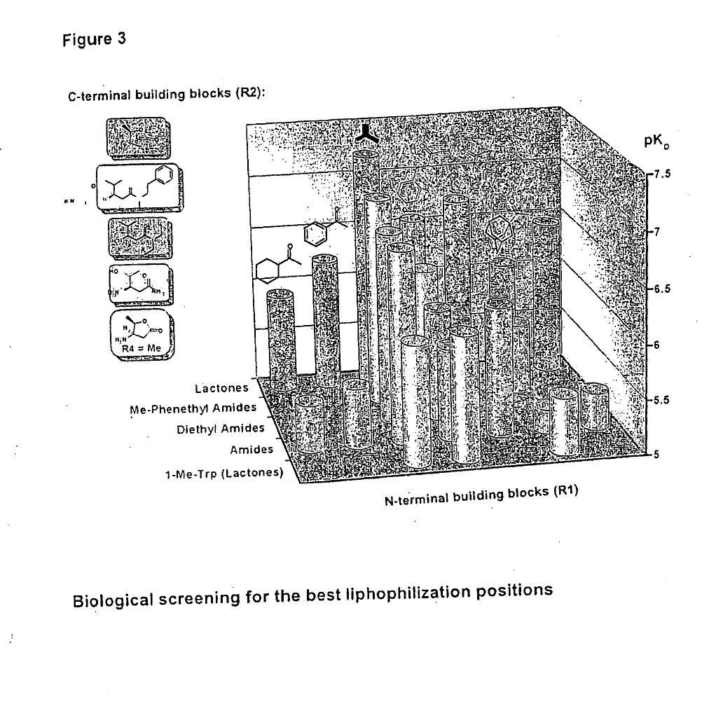

Replacement of (R)-tryptophane for N-Me-indol-modified (R)-tryptophane (R⁴=Me in scaffold I) within the collection of the more potent C-terminal β-homothreonine-lactones (see row 2 in Table 1) provides ligands with decreased hsst4 binding affinities. The potency of these compounds (20, 19 and 21) was somehow situated between those of the β-homothreonine-lactones and the ones of the β-homothreonine-amides (see pale yellow columns in FIG. 3) having K_{D} values from 708 nM (compound 19) to 2951 nM (compound 20). No expected significant changes in membrane permeability and solubility are observed with this approach.

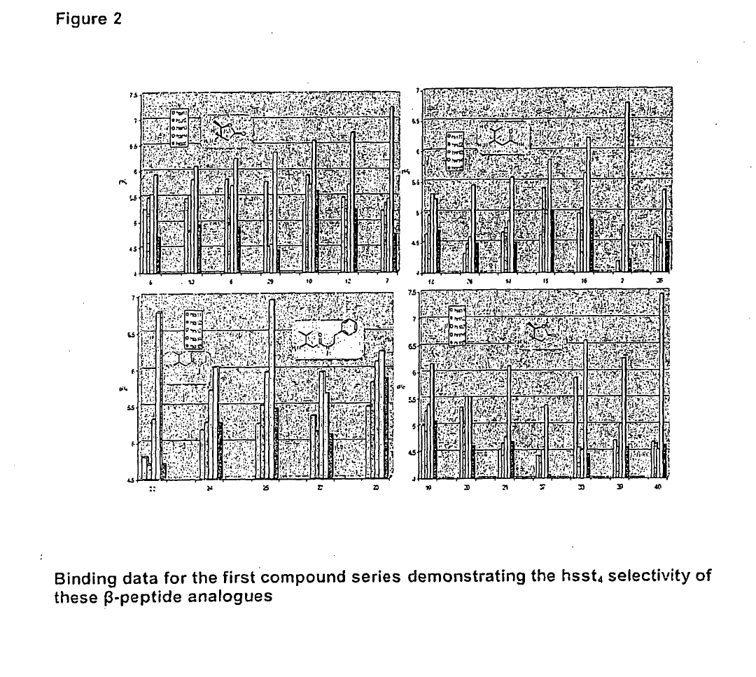

Prolongated analoges having C-terminal modified β-leucin-methyl-phenethyl-amides (compounds 25, 27 and 23 in Table 1) or β-leucin-diethyl-amides (compounds 22 and 24 in Table 1) instead of β-homothreonine amide show similar (166 nM for compound 22), some of them even improved binding affinities (115 nM for compound 25) and selectivities to the hsst4 receptor (see FIGS. 2 and 3). Changes at the N-terminal positions are more pronounced than on the C-termini and suggests that only linear (non-branched) lipid residues might have a chance as useful N-terminal liphophilization tags.

The studies with β-homothreonine amides, β-leucine amides and 4-amino-5-methyl-dihydro-furan-2-ones (β-homothreonine lactones) clearly show that several functional groups are not necessarily important for high affinity binding to the hsst4 receptors. For instance, the hydroxy group of the β-homothreonine can be replaced by a simple methyl residue without losing binding affinity (compare K_{D} values of compound 22 with compounds 1 to 3). The amide functionality is obviously without significant binding function as the lactone based compounds 7 to 9 show much higher binding affinity than all of the corresponding open-chained amides (compounds 1 to 3, 22 and 25). From the biological data it is not evident whether the beta-turn, formed in the sequence Ac-(S)-β³-HPhe-(R)-Trp-(S)-β³-HLys-(R)-4-amino-(R)-5-methyl-dihydro-furan-2-one, is stabilized through intramolecular hydrogen bonding as it was described in literature by Gademann et al (2001). Especially the C-terminal carbonyl functionalities (lactones 7 to 9 vs amides 1 to 3, 25) are significantly different in their structural arrangements. From this, an involvement in intramolecular hydrogen bonding is not obvious.

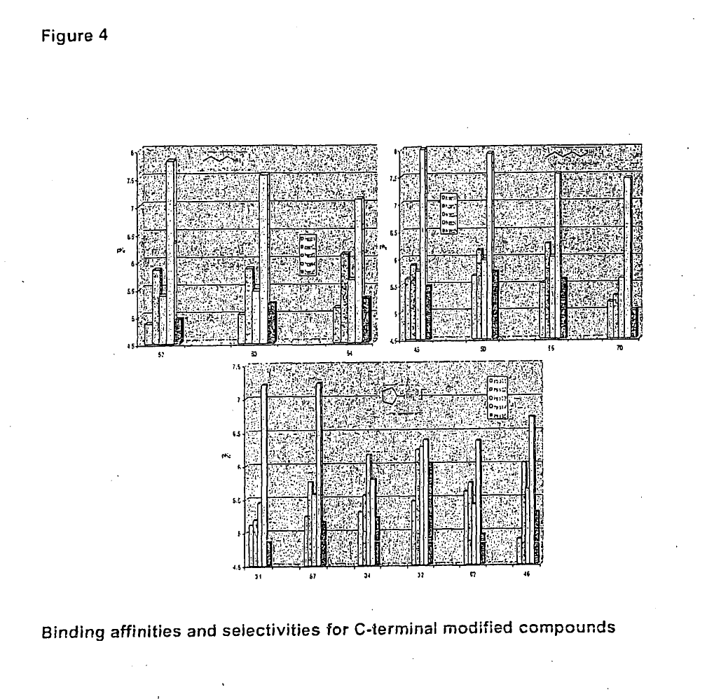

Testing of the established C-terminal cyclopentyl β-homolysine amides (see row five in Table 1) affords an exact match with the biological data derived from the compound collection in which β-homothreonine lactone is in C-terminal position. For comparison see e.g. compounds 7 to 9, a 60 nM ligand on hsst4 with compound 30 having a K_{D} of 62 nM for the same receptor. The N-terminal exchange of the acetyl-group for branched analogues leads to a decrease in binding affinity and for some members in selectivity as well (see FIG. 4).

Derivatization with e.g. hydrocinnemoyl chloride affords a ligand (see compound 32) with moderate binding, affinity to the whole SRIF-1-receptor family. Although the potency of this ligand (417 nM) is lower compared to the N-acetyl congener (62 nM), this might be a good starting point for the synthesis of further β-peptide based somatostatin analogs having a universal binding profile.

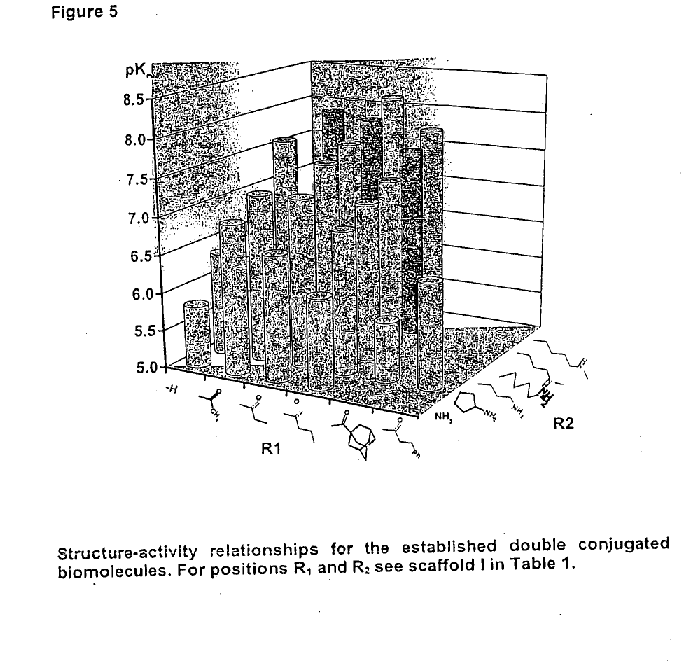

Linear lipophilization tags are tolerated best on the N-terminal peptide position. Activities are slightly decreasing through homologous prolongation of the N-terminal tail. This applies for most of the tested compounds with some exceptions having highest binding affinity when N-terminally capped with a propionyl residue (see FIG. 5).

Modifying the non-decorated scaffold structure of compound 59 ((S)-β³HPhe-(R)-Trp-(S)-β³-HLys-NH₂) at the C-terminus with linear (non-branched) lipophilization tags (see FIGS. 4 and 5) leads to more active compounds. These modifications not only bring compounds with improved activities (e.g. K_{D}=16 nM for compound 52 and K_{D}=10 nM for compounds 44 and 45) but have a positive impact on physicochemical properties, especially on overall hydrophobicity. According to Lipinski's rule of five, In Silico profiling of the established conjugated biomolecules shows drug-like properties with one major violation class, the number of hydrogen bond donors.

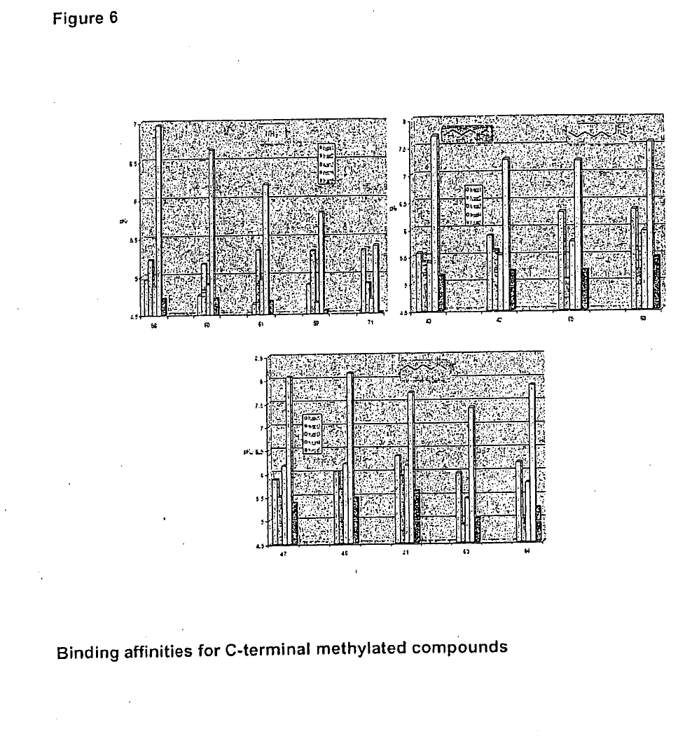

Introduction of a simple methyl group at the C-terminal (S)-β-homolysine-butyl and pentyl amides giving compounds 43, 42 or 47, 48, 49 and 41 is fully compatible with the binding profile and led in all cases to an increase in binding affinities (e.g. K_{D}=7 nM for compounds 48 and 49) and to high selectivities (see FIGS. 5 and 6).

N-terminal exchange of the hydrogen atom for a methyl group gives ligands with lower binding affinities. This applies for N-acylated and N-propionylated (e.g. compounds 39 and 46) compounds as well as for non-acylated N-aminomethyl-(S)-β-homophenylalanine analogues (compound 38).

Monomethylation of the amino acid on the primary amine functionality of the (R)-tryptophane moiety (see compounds based on scaffold II in Table 2) and subsequent incorporation on the dedicated position within the peptide give hsst4 selective ligands with binding affinities ranging from 57 nM (for compound 65) to 35 nM (for compound 70). Depending on the C-terminal residue this slight modification in the backbone affords peptides with remarkable selectivies up to a factor 1000 amongst other hsst receptors (e.g. compound 40). Furthermore, with N-monobenzylation at the same position even higher binding affinities with K_{D} values as low as 14 nM (for compounds 66 and 67) can be achieved. These ligands are less selective towards hsst1 receptors, but still by a factor 100 selective amongst other hsst receptors.

hsst4 selectivity of mixed α/β³-peptides might therefore be controlled through the selection of appropriate C-terminal amide residues in combination with N-amino alkylated (R)-tryptophane building blocks as highlighted for scaffold II (see Table 1). This stands in good correlation with the general finding that the basic scaffold of compound 59 ((S)-β³-HPhe-(R)-Trp-(S)-β³-HLys-NH₂) (shown in Table 1) has only very low affinity to all of the receptors of the SRIF family (e.g. 1514 nM for hsst4), but can be transformed into highly potent and hsst4-selective ligands through distinct structural manipulations at the C-terminal, N-terminal and backbone positions.

### Physicochemical Properties of the Compounds

To reach the therapeutic target site, a molecule must permeate through many natural barriers formed by cell membranes. These are composed of phospholipid bilayers—oily barriers that greatly attenuate the passage of charged or highly polar molecules. Accompanied with the fast proteolytic degradation this is the biggest disadvantage for drugs based on peptide structures.

To be absorbed and transported by passive diffusion, drugs must be sufficiently lipophilic to penetrate the lipid cores of membranes, but not too lipophilic that they get retained and accumulated there. The lipophilictity of a compound is expressed by the octanol/water partition coefficient or distribution coefficient. A first approximation of substance polarity can either be given by computer assisted calculations giving Clog P values or by measurement of the partition coefficients in high-throughput assays (HT-log P o/w) (Faller et al., 2004; Wohnsland et al., 2001).

Several high throughput assays for the determination of physicochemical properties have been established. Some of these works have particularly focused on the development of high-throughput test systems providing accurate and reproducible values of octanol/water partition (log P) and distribution coefficients. Values derived from these approaches have proven to be useful parameters for the estimation of lipophilicity and polarity of compounds.

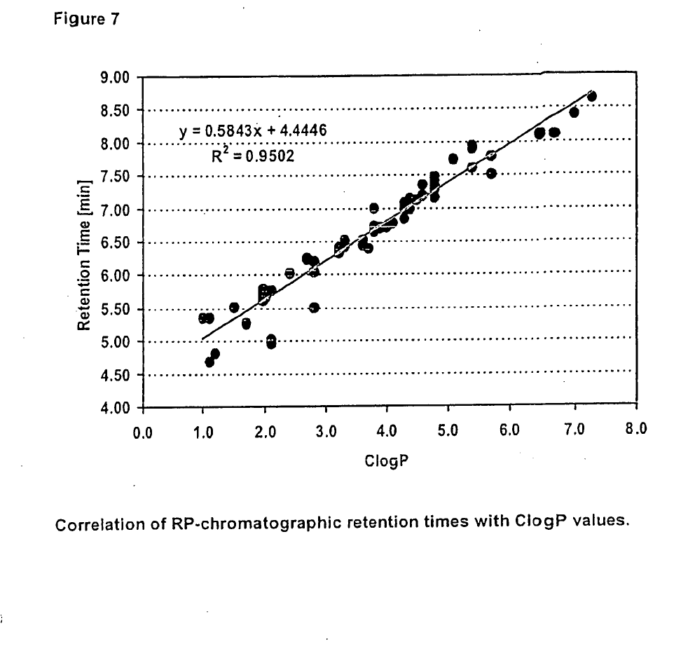

The calculated partition octanol/water coefficients (ClogP) of the lipopeptides of the invention stand in good correlation with RP-chromatographic retention times (see FIG. 7).

Overall substance polarity is mainly driven by the introduced lipophilic residues. Combinations of bulky N-terminal residues (adamantane, nonanoyl) with lipophilized β-amino acid building blocks at the C-terminal position (for examples see compounds 23, 24 or 27) gives long retention times and provides ClogP values up to 7. The other extreme on the polarity scale is represented by the non-substituted tripeptidic scaffold structures of compounds 59 and 72 which provide shortest run times and had ClogP values below 2. Introduction of small linear capping groups at the N-terminal scaffold position (e.g. compounds 60 and 61) bring a slight increase of Clog P values and in retention times. The same applied for C-terminal modifications, whereas backbone modification with β-homothreonine amide (see compounds 36 or 2) or 4-amino-5-methyl-dihydrofuran2-one (compounds 29 or 7) do not significantly contribute to the reduction of the overall polarity.

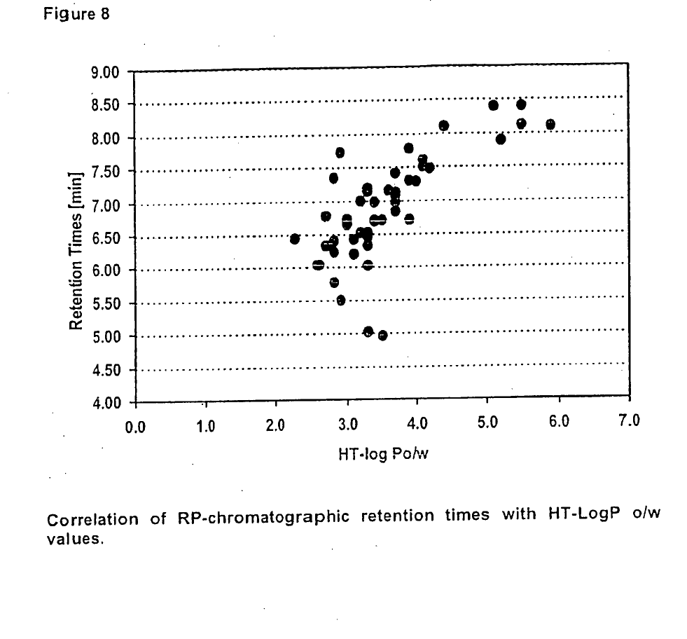

Octanol/water partition coefficients are measured in a high throughput assay based on artificial liquid membrane permeability. Comparison of the measured with the calculated values clearly demonstrated that only low or almost no correlation does exist (see FIG. 8). Most of the calculated values are significantly overestimating the measured values. This can be attributed to the fact that the calculations are based on linear fragment increments but do not consider folding into secondary peptide structures. Still many approaches to calculate log P values are limited due to a lack of parameterization of certain fragments and fail with increasing molecular weight which implicates higher structural complexity. Similar problems and failures are observed in chromatographic determinations of log P: Peptides might undergo structural changes driven by interactions with the stationary phase. Decisive interactions between the stationary phase and the defolded analytes are therefore also based on the linear peptide sequence. For this reason measured HT-log P o/w values based on chromatographic assays employing stationary phases may lead to erroneous results.

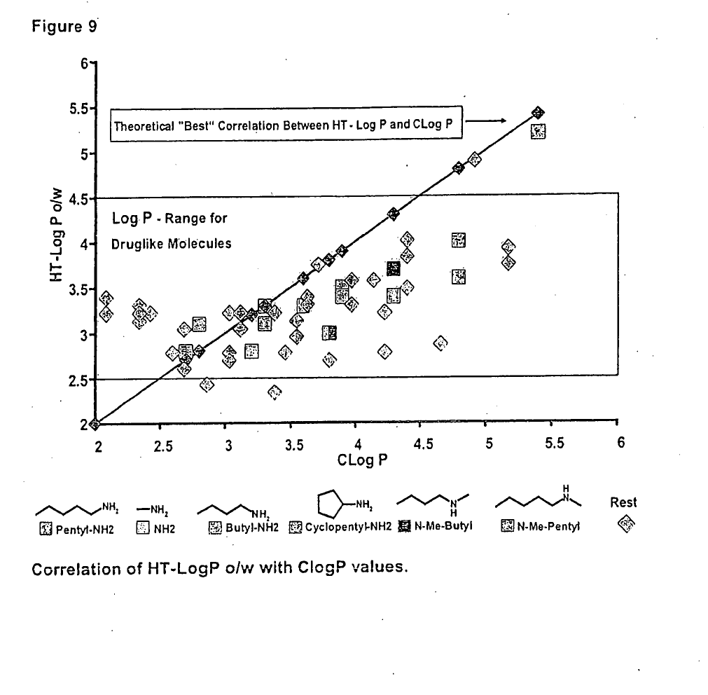

From FIG. 9 can be seen that the selection of lipophilic residues gives an ideal distribution over the whole range proposed for druglike molecules (log P=2.5-4.5) (Corner, 2003). Partition coefficients for highly polar compounds (2, 7, 36, 58, 59, 60 and 61) can not be determined in the high throughput assays based on artificial membranes (primary assays) since determinations of log P values with appropriate accuracy are limited to values above 2 in this specific test setup. As calculated partition coefficients (Clog P) give numbers between 1 and 2, and by following the general trend line in the corresponding HT-logP/Clog P correlation diagram (FIG. 9), it can be estimated that the log P values of these compounds are close to or even below 0.

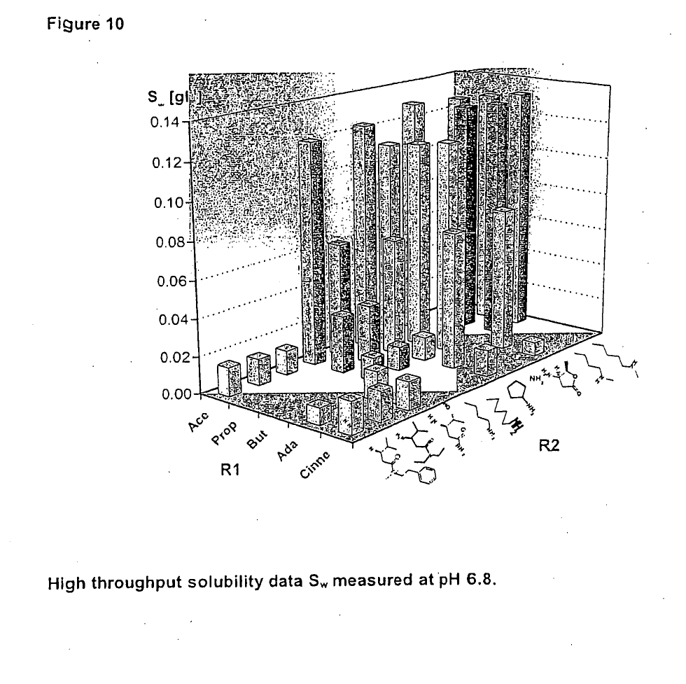

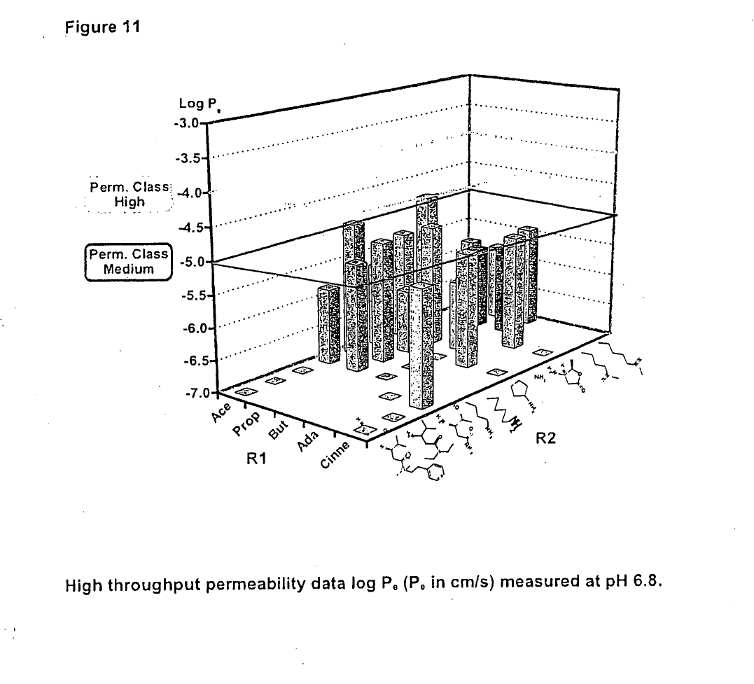

In order to have a real measure of log P values for highly polar compounds, respective values for compounds 1 to 3 (C-terminal L-β-homothreonine amides) and compounds 7 to 9 (C-terminal 4-amino-5-methyl-dihydro-fuan-2-ones) are measured by employing pH-metric titration technology using a GLpKa instrument (Box et al. 2003). The high polarity of these two mixed α/β³-tetrapeptides can be controlled by N-terminal introduction of lipophilization tags. The conjugation with fatty acid analogs leads to compounds with druglike polarity (5, 6, 10, 11, 12, 13, 14, 15, 16, 17, 18 and 26). Only very few members (e.g. compound 13) of this first series of conjugated biomolecules show good water solubility accompanied with acceptable permeability profiles (see FIGS. 10 and 11).

The series based on the more polar mixed α/β³-tetrapeptide N-terminal modified β-homothreonine amides (compounds 1 to 3) shows a very unsatisfactory physicochemical profile. This might be taken as a proof that the solubility is not only dependent on compound polarity or lipophilicity alone but is strikingly influenced by the number of hydrogen bond donors and/or acceptors of a substance. As with an increased number of hydrogen bond acceptors and/or donors more intermolecular interactions might occur, consequently increased compound agglomeration should be observed. This stands in good accordance with the fact that a general decrease in solubility was found by going from the series based on C-terminal β-homothreonine lactones (compounds 7 to 9) (respective derivatives 5, 6, 10, 11, 12, 13) to the C-terminal β-homothreonine amide based derivatives (14, 15, 16, 17, 18, 26).

The N-Methylation at the tryptophane indole leading to a further reduction of the number of hydrogen bond donors and acceptors affording substances 19, 20 and 21, did not lead to any improvements concerning solubility or permeability. A reason for this interesting finding might be that not all of the donors or acceptors have the same influence on physicochemical characteristics.

In the series of compounds 22 to 25 and 27 C-terminal β-homothreonine amide is replaced by β-leucine amides. The introduction of secondary amides brings a reduction of hydrogen bond donors and is accompanied with overall lipophilization. Although the strategy is in good accordance with biological test results (see there), the further increase in molecular weight through derivatization does not allow improvements in permeability. Apart from a high polar surface area, a large molecular weight is another limiting factor in cell permeation of compounds. These highly lipophilic conjugated biomolecules are of poor water solubility.

Initial structure activity relationship (SAR) studies have demonstrated that some of the groups contributing to the large number of hydrogen bond donors and acceptors are not involved in hsst receptor binding recognition (for details see biological test results) and can therefore be replaced by other structural motifs, for example by introduction of a cyclopentyl ring for mimicking the C-terminal dihydro-furan-2-one unit.

The resulting compound series 30 to 34, 46, 56, 57 and 62) is taking profit of a lower number of hydrogen bond acceptors which can be decreased from 12 to 10. As a consequence solubilities are in the range between medium and good for most of these substances (see FIG. 10). Additionally, the lower molecular weight and the decreased polar surface area allowed for moderate membrane permeabilities.

The C-terminal cyclopentyl fragment is exchangeable for other linear lipophilization tags. This double conjugation gives the opportunity to regulate the logP values from both the N-terminal as well as from the C-terminal peptide position, and in best case scenarios it is possible to find the right equilibrium between permeability and solubility. An optimum balance between these two decisive physicochemical characteristics can be found for compounds having logP values between 2.8 and 3.8 especially when focusing on drugs with their mode of action in the central nervous system (CNS). As can be seen from FIG. 6, all of these double conjugated biomolecules (see compounds 44, 45, 50, 51, 52, 53, 54 and 55) hit the desired logP range for drug like molecules. In analogy to the C-terminal cyclopentyl analogs, solubilities range from medium to good with only a few exceptions, namely compounds 54 and 55, which might be attributed to the higher lipophilicity of these N-butyroylated compounds. In addition, also the membrane permeability measures for these substances are much more satisfying when compared to previous compound series.

Further improvements can be achieved as the number of hydrogen bond donors of the compounds above is still at a value of 7, thus violating the rule of five criteria for drug like molecules (≦5 HBD). A subsequent methyl scan through the amide backbone shows with substances still having high binding affinity values to hsst4 receptors, but fulfilling the aforementioned criteria. Respective compounds having only five to six hydrogen bond donors have excellent solubility values. This might also be attributed to the elevated imbalance between hydrogen bond donors (5 or 6) and acceptors (10). It has been shown by theoretical calculations (Abraham et al., 1999) and in some practical examples (Faller, 2003) that the creation of an imbalance between hydrogen bond-donors and acceptors through reduction of donor numbers can bring an increase in solubility. In fact, this applied for all of the investigated N-methylated double conjugated biomolecules (41; 42; 43; 47; 48; 49, 63, 64, 65 and 70) having highest solubility values amongst all other substances. Exceptions are found for compounds 66, 67 and 68, 69 (N-benzylated double conjugated biomolecules). Although fulfilling hydrogen bond donor and acceptor criteria, the lipophilicity of these compounds manifested in high logP values does not allow for good water solubilities. The same was found for membrane permeabilities which might be more related to the large molecular weight of these two substances. N-Methylated conjugated biomolecules (e.g. 63, 64, 65, 70 and 48, 49) showed some medium permeability (see FIG. 11).

### General Experimental Procedure

The following general experimental procedures described below were used for the synthesis of all of the compounds of the present invention.

### Purification Representative Procedure:

### Preparative LC/MS System:

The preparative HPLC/MS system was consisting of a Waters 600 quaternary pump, a 233 XL injector from Gilson, a 215 fraction collector from Gilson and a 2487 UV detector from Waters. The preparative column was a Xterra MS C18 5 μm, 19×100 mm column. Mobile phases A: water (0.1% TFA), B: acetonitrile (0.1% TFA). A typical gradient was 2% B for 1.0 min then to 95% B within 8 min, 95% B for 1 min then back to 2% B. Total run time 10 min. UV-signal at 214 nm, Flow from 15 ml/min to 30 ml/min within first minute of run, Temp: ambient. The MS signal was measured with a platform from Micromass (ZMD mass detector). The operating conditions in ESI⁺ mode were the following: source block temperature, 120° C.; desolvation temperature, 200° C.; ion energy, 1.0 V; capillary voltage 3.5 kV; cone voltage, 20 V; extractor, 3 V. The samples were dissolved in DMA/(Water/TFA=4/1)=4/1, and an amount of 900 μl of solution was injected.

### Preparative LC/UV System:

The preparative LC/UV system was consisting of a preparative pump from SepTech, a UV spectrophotometer from Labomatic and an Asted XL fraction collector from Gilson, The preparative column was a Nucleodur 100-10 C18 ec column from Macherey-Nagel. Mobile phase: acetonitrile 0.1% TFA/water 0.1% TFA. The gradient was starting with 90% water and finishing at 90% Acetonitrile within 15 min; Detection: UV 215 nm. The samples were dissolved in DMSO, and an amount of 1 ml of solution was injected.

### Analytical HPLC was Typically Carried Out in the Following Systems:

System I (Merck Hitachi): Solvent A was water (0.1% TFA) and Solvent B was acetonitrile (0.1% TFA). The gradient was 5% B to 95% B within 10 min, 2 min at 95% B then immediately back to 5% B and equilibration for 3 min at 95% A. Total run time: 15 min at a flow rate of 0.8 ml/min. Column: MN Nucleosil (100-3, RP-C-18 from Macherey and Nagel). Temperature: 40° C., UV detection at 220 nm. The samples were dissolved in ACN (0.1% TFA)/Water (0.1% TFA)=75/25, and an amount of 10 μl of solution was injected.

System II (Waters Alliance 2795): Solvent A was water (0.1% TFA) and Solvent B Was acetonitrile (0.1% TFA). The gradient was 5% B to 100% B within 10 min, 0.5 min at 100% B then immediately back to 5% B and equilibration for 1.5 min at 95% A. Total run time: 12 min at a flow rate of 0.8 ml/min. Column: MN Nucleosil (100-3, RP-C-18 from Macherey and Nagel). Temperature: 40° C., UV-DAD detection at 220 nm, 254 nm, PDA Max Plot (210 nm to 400 nm). The samples were dissolved in ACN (0.1% TFA)/Water (0.1% TFA)=75/25, and an amount of 10 μl of solution was injected.

### General Procedures:

General Procedure for Coupling of β-Amino Acids with TBTU and HOAt/HOOBt (Gademann et al., 2000):

The hydrochloride of the amino fragment and the Boc-protected fragment (1 equiv.) were suspended in a mixture of anhydrous dichloromethane and anhydrous dimethylformamide (3/1) (0.2 M) at room temperature under argon. After cooling to 0° C. (ice/water), TEA (5 equiv.) was added, and the resulting mixture was stirred at 0° C. for 15 min under argon. Then, HOAt or HOOBt (1.2 equiv.) were added and stirring was continued for 15 min. Finally, TBTU (1.2 equiv.) was added and the mixture was stirred at r.t. for 12 h. Dilution with DCM was followed by extraction with a solution (5%) of NaHCO₃/NaCl and sat. NaCl, subsequently with 1 M citric acid and finally again with saturated NaCl. The organic layer was dried (Na₂SO₄), removed under reduced pressure and the resulting solid residue used for the next step without any further purification. N-Me beta amino acids were much better soluble than the non-methylated analogues.

General Procedure for Coupling of β-Amino Acids with HATU and HOAt:

The hydrochloride of the amino fragment and the Boc-protected fragment (1 equiv.) were suspended in a mixture of anhydrous dichloromethane and anhydrous dimethylformamide (3/1) (0.2 M) at room temperature under argon. After cooling to 0° C. (ice/water), sym.—Collidine (10 equiv.) was added, and the resulting mixture was stirred at 0° C. for 15 min under Argon. Then, HOAt (1.2 equiv.) was added and stirring was continued for 15 min. Finally, HATU (1.2 equiv.) was added and the mixture was stirred at r.t. for 16 h. Dilution with DCM was followed by extraction with 1 M citric acid and sat. NaCl, subsequently with NaHCO₃/NaCl and finally again with saturated NaCl. The solvent was removed under reduced pressure and the resulting solid residue used for the next step without any further purification.

### General Procedure for the Boc-Protection of Amino Acids (Levy et al., 1998):

The amino acid was dissolved in dry DMF (1 g/10 ml), and triethylamine (3 equiv.) was added followed by di-tert.-butyl dicarbonate (1.2 equiv.). The reaction mixture was stirred at room temperature for 15 h after which it was concentrated to dryness and the residue was dissolved in EtOAc. The resulting mixture was washed with saturated NaHCO₃. The combined aqueous extracts were acidified to pH=3 (pH paper) with 6 N HCl and washed with EtOAc. The combined organic extracts were dried over anhydrous MgSO₄, filtered and concentrated to give the desired product. The product was used for the next step without further purification.

Boc-Deprotection with HCl in 1,4-Dioxane:

The Boc-protected compound was suspended in 1,4-dioxane (0.2 M) and treated with a solution of hydrogen chloride in 1,4-dioxane (40 equiv.). The resulting solution was stirred at r.t. for 90 min. The volatile components were removed under reduced pressure, the resulting residue dried under high vacuum and used for the next step without further purification.

Boc-Deprotection with Formic Acid:

The Boc-protected compound was dissolved in formic acid (200 equiv.) and stirred at r.t. for 45 min (LC/MS control). Immediately after the reaction reached completeness, the product solution was diluted with toluene and the solvents removed in vacuum. This procedure was repeated three times and the residue then dried in high vacuum to give the formic acid salt of the desired product. In order to remove the formic acid (partial formylation was observed during next coupling step when the formate of the amino fragment was used), the crude product was suspended in DCM and pre-activated (activation by standing in 6% TEA in DCM (3×30 min)) D-series lanterns (provided by Mimotope, www.mimotopes.com) containing aminomethyl linkers (loading: 100 μmol/lantern) were added. The mixture was then slightly stirred at r.t. for 12 h. The lanterns were then removed and washed several times with DCM and MeOH. The solvents were removed in vacuum and the product dried in high vacuum to give formic acid free product.

### Pd-Catalyzed Z-Deprotection by Hydrogenolysis:

The Z-protected compound was suspended in a solution of TFA (10%) in DMA (0.25 M). Then palladium on activated charcoal (10%) (30 mg/0.1 mmol) was added and the resulting reaction mixture stirred under hydrogen (1 balloon) (1 balloon/0.3 mmol of substrate) at r.t. for 5 h. The crude reaction mixture was freed from charcoal by filtration through HPLC filters (Gelman Acrodisc PTFE membrane 0.2 μm) and the volatile components were removed in vacuum. The solid residue was then purified by reversed phase chromatography (see general procedure).

### N-Methylation of Boc-β-Homophenylalanine (Gademann et al., 2000):

Boc-L-β-homophenylalanine (500 mg; 1.79 mmol) was dissolved in THF (18 ml; 0.1 M), MeI (900 μl, 8 equiv.) was added, the solution cooled to 0° C., and NaH (60% oily suspension, 215 mg; 3 equ.) was added in portions. The mixture was allowed to warm up to r.t. and stirred for 22 h, then cooled to −10° C. and excess NaH was hydrolized with ice. The solvents were evaporated, and the residue dissolved in water (20 ml). The aqueous phase was washed with diethylether (15 ml) (the pH was adjusted to ca. 2 with sat. aqueous KHSO₄ soln., few drops) and extracted with diethylether (3×20 ml). The organic phase was washed with 0.5 M HCl solution (3×10 ml) and dried (MgSO₄). The solvent was removed under reduced pressure to yield Boc-protected N-Methyl-β-homophenylalanine (468 mg, 89%) which was used without further purification.

### N-Methylation of N-Boc-1-Me-(R)-Tryptophane:

Boc-1-Me-(R)-tryptophane (380 mg; 1.19 mmol) was dissolved in THF (12 ml), MeI (594 μl, 8 equiv.) was added the solution cooled to 0° C., and NaH (149 mg; 3 equiv.) was added in portions. The mixture was allowed to warm up to r.t. and stirred for 22 h, then cooled to −10° C. and excess NaH was hydrolized with ice. The solvent was evaporated, and the residue was dissolved in water (20 ml). The aqueous phase was washed with diethylether (15 ml) (the pH was adjusted to ca. 2 with sat. aqu. KHSO₄ soln., few drops) and extracted with diethylether (3×20 ml). The organic phase was washed with 0.5 M HCl soln. (3×10 ml) and dried (MgSO₄). The solvent was removed under reduced pressure to yield Boc-N-methyl-1-methyl-(R)-tryptophane (350 mg; 88%) which was used in the next step without further purification.

### N-Methylation of N-Boc-1-Boc-(R)-Tryptophane:

N-Boc-1-Boc-(R)-tryptophane (2 g; 4.94 mmol) was dissolved in THF (25 ml), MeI (2.46 ml, 8 equiv.) was added and the solution cooled to 0° C. Then NaH (356 mg, 60% oily suspension; 3 equ.) was added in portions. The mixture was allowed to warm up to r.t. and stirred for 36 h under nitrogen. Then, ethyl acetate (20 ml) was added, followed by water. The solvents were evaporated to dryness, and the oily residue partitioned between ether so (2×25 ml) and water (100 ml). The ether layer was washed with aqueous NaHCO₃ (2×25 ml), and the combined aqueous extracts acidified to pH 3 with sat. aqueous KHSO₄ solution (ca. 60 ml). The product was extracted with ethyl acetate (3×30 ml), the organic layer washed with water (2×30 ml), 5% aqueous sodium thiosulfate (2×30 ml; to remove iodine), water (30 ml) and dried over MgSO₄. The solvent was removed under reduced pressure to yield N-Boc-N-methyl-1-Boc-R-tryptophane (1.5 g, 72% crude) as yellowish oil which was crystallized from ethyl acetate, but was further purified by column chromatography (DCM/MeOH=10/1; silica: 150 g) to give a white powder (1.01 g; 75:25 mixture of two products as determined by analytical reversed phase HPLC). The mixture was therefore subsequently purified by preparative reversed phase chromatography (Agilent 1100 series prep instrument, column: Waters, Xterra prep RP₁₈ OBD Column, 5 μm, 19×50 mm, A: Water (0.1% TFA), B: Acetonitrile (0.1% TFA) Gradient: 30% B for 1.5 min to 100% B within 7 min, 100% B for 1 min back to 30% B, total run time: 10 min, UV-DAD signal at 220 nm, Flow 20 ml/min, Temp: r.t.) to give a pure amorphous white powder (750 mg, purity >99%).

### N-Benzylation of D-Tryptophane (Quitte et al. 1963):

(R)-tryptophane (1 g; 4.9 mmol) was suspended in 2 N NaOH (25 ml) and mixed under permanent stirring with benzaldehyde (500 μl; 4.90 mmol). The resulting solution was stirred at r.t. for 30 min and subsequently treated with sodium cyanoborohydride (92 mg; 1.47 mmol). Addition was carried out in small portions in order to keep the temperature below 15° C. After addition was completed the resulting suspension was stirred at room temperature for additional 30 min and the whole procedure (benzaldehyde, sodium cyanoborohydride) repeated. The resulting mixture was stirred overnight at room temperature (16 h). Since there was still some starting material left on the next day, the procedure was repeated with the same amounts of benzaldehyde and sodium cyanoborohydride. The mixture was then stirred at r.t. for another 16 h. The reaction mixture was washed with diethyl ether and neutralized (pH 6-7) with 1 N HCl under vigorous stirring. The benzyl-amino acid precipitated immediately, was filtered, washed with water (3×20 ml) and dried under vacuum to give of a slightly yellowish amorphous solid (750 mg; 52%). The product was used in the next step without further purification.

### List of Compounds

The following compounds of the invention were produced and analysed with the previously described experimental methods which are known to the skilled person. Yield, purity as determined in LC-MS-UV reversed phase analytical experiments, Rt and HRMS data are given below for each compound. Where relevant, compounds were analyzed with ¹H and ¹³C-NMR.

### Ac—(S)-β³-HPhe-(R)-Trp-(S)-β³-HLys-(R), (R)—HThr-NH₂ (Compound 2)

15 mg, >94% purity, Rt=5.22, HRMS [M+H]⁺ 664.3816 (calcd 664.3817).

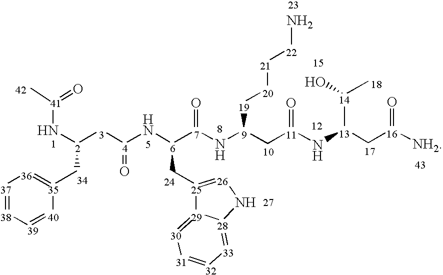

¹H (500 MHz, DMSO): δ (ppm)=0.95 (d, 3H, C₁₈H3); 1.20 (m, 2H, C₂₀H2); 1.28 (m, 2H, C₁₉H2); 1.38 (m, 2H, C₂₁H2); 1.6 (s, 3H, C₄₂H3); 2.12, 2.35 (m, 2H, C₁₇H2); 2.25 (m, 4H, C₃H2, C₁₀H2); 2.6 (m, 4H, C₂₂H2, C₃₄H2); 2.9, 3.08 (m, 2H, C₂₄H2); 3.65 (m, 1H, C₁₄H); 3.95 (m, 1H, C₉H); 4.02 (m, 1H, C₁₃H); 4.2 (m, 1H, C₂H); 4.52 (m, 1H, C₆H); 4.68 (m, 1H, O₁₅H); 6.72, 7.08 (m, 2H, N₄₃H2); 6.96 (m, 1H, C₃₁H); 7.0-7.1 (m, 2H, C₃₂H, C₂₆H); 7.11 (m, 2H, C₃₆H, C₄₀H); 7.15 (m, 1H, C₃₈H); 7.2 (m, 2H, C₃₇H, C₃₉H); 7.3 (d, 1H, C₃₃H); 7.45 (d, 1H, N₁₂H); 7.55 (m, 1H, C₃₀H); 7.12 (m, 1H, N₁H); 7.72 (d, 1H, N₈H); 8.05 (d, 1H, N₅H); 10.8 (s, N₂₇H).

¹³C (125 MHz; DMSO): δ (ppm)=20.1 (C₁₈H3), 22.4 (C₂₀H2), 23.1 (C₄₂H3), 27.1 (C₂₁H2), 28.5 (C₂₄H2), 33.1 (C₁₉H2), 37.0 (C₁₇H2), 39.2 (C₂₂H2), 40 (C₃₄H2), 40.5 (C₁₀H2), 41.2 (C₃H2), 46.4 (C₉H), 48.3 (C₂H), 51.3 (C₁₃H), 54.3 (C₆H), 67.0 (C₁₄H), 110.6 (C₂₅), 111.7 (C₃₃H), 118.6 (C₃₁H), 118.8 (C₃₀H), 121.3 (C₃₂H), 123.8 (C₂₆H), 126.4 (C₃₈H); 127.7 (C₂₉), 128.5 (C₃₇H, C₃₉H), 129.6 (C₃₆H, C₄₀H), 136.5 (C₂₈), 139.2 (C₃₅), 168.9, 170.1 (C₄═O), 170.4 (C₁₁═O), 171.3 (C₇═O), 173.1.

### Cyclohexanoyl-(S)-β³-HPhe-(R)-Trp-(S)-β³-HLys-(R)-4-amino-(R)-5-methyl-dihydro-furan-2-one (Compound 5)

13 mg, >99% purity, Rt=6.70, HRMS [M+H]⁺ 715.4180 (calcd 715.4178).

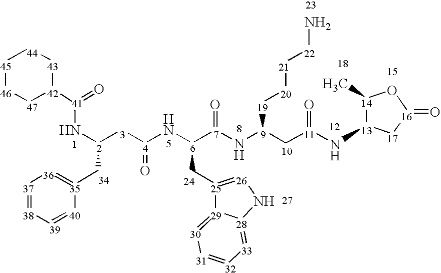

¹H (500 MHz, DMSO): δ (ppm)=1.0 (m, 2H, C₂₀H2); 1.1 (m, 5H, C₁₈H3, C₄₅H2); 1.12, 1.52, 1.6 (m, 4H, C₄₃H2, C₄₇H2); 1.2 (m, 2H, C₁₉H2); 1.4 (m, 2H, C₂₁H2); 1.5 (m, 4H, C₄₄H2, C₄₆H2); 1.9 (m, 1H, C₄₂H); 2.2 (m, 4H, C₃H2, C₁₀H2); 2.3 (m, 1H, C₁₇H2); 2.6 (m, 3H, C₃₄H2, C₂₂H2); 2.7 (m, 1H, C₃₄H2); 2.85 (m, 1H, C₂₄H2); 2.9 (m, 1H, C₁₇H2); 3.1 (m, 1H, C₂₄H2); 4.0 (m, 1H, C₉H); 4.2 (m, 1H, C₂H); 4.5 (m, 2H, C₆H, C₁₃H); 4.7 (m, 1H, C₁₄H); 6.9 (m, 1H, C₃₁H); 7.0 (m, 1H, C₃₂H); 7.1 (m, 2H, C₃₆H, C₄₀H); 7.12 (m, 1H, C₂₆H); 7.2 (m, 2H, C₃₇H, C₃₉H); 7.3 (m, 1H, C₃₃H); 7.5 (m, 1H, N₁H); 7.6 (m, 1H, C₃₀H); 7.62 (br s, 3H, N₂₃H3⁺); 7.8 (d, 1H, N₈H); 8.1 (d, 1H, N₅H); 8.3 (d, N₁₂H); 10.8 (s, N₂₇H).

¹³C (125 MHz; DMSO): δ (ppm)=48 (C₂H), 41 (C₃H2), 170.4 (C₄), 54.1 (C₆H), 171.3 (C₇), 46.1 (C₉H), 40.8 (C₁₀H2), 170.6 (C₁₁), 48.7 (C₁₃H), 79.2 (C₁₄H), 175.7 (C₁₆), 35.6 (C₁₇H2), 15 (C₁₈H3), 33.3 (C₁₉H2), 22.4 (C₂₀H2), 27.2 (C₂₁H2), 39.2 (C₂₂H2), 28.5 (C₂₄H), 110.6 (C₂₅), 123.8 (C₂₆H), 136.5 (C₂₈), 127.7 (C₂₉), 118.8 (C₃₀H), 118.6 (C₃₁H), 121.3 (C₃₂H), 111.7 (C₃₃H), 40 (C₃₄H2), 139.3 (C₃₅), 129.7 (C₃₆H, C₄₀H), 128.4 (C₃₇H, C₃₉H), 126.4 (C₃₈H); 174.8 (C₄₁), 44.5 (C₄₂H), 29.6 (C₄₃H2), 25.6 (C₄₄H2), 25.6 (C₄₅H2), 25.7 (C₄₆H2), 29.4 (C₄₇H2).

### Benzoyl-(S)-β³-HPhe-(R)-Trp-(S)-β³-HLys-(R)-4-amino-(R)-5-methyl-dihydro-furan-2-one (Compound 6)

22 mg, >91% purity, Rt=6.39, HRMS [M+H]⁺ 709.3710 (calcd 709.3708), NMR (¹H; ¹³C).

### Ac—(S)-β³-HPhe-(R)-Trp-(S)-β³-HLys-(R)-4-amino-(R)-5-methyl-dihydro-furan-2-one (Compound 7)

25 mg, >99% purity, Rt=5.65, HRMS [M+H]⁺ 647.3550 (calcd 647.3552), NMR (¹H; ¹³C).

### Dihydrocinnemoyl-(S)-β³-HPhe-(R)-Trp-(S)-β³-HLys-(R)-4-amino-(R)-5-methyl-dihydro-furan-2-one (Compound 10)

13 mg, >99% purity, Rt=6.77, HRMS [M+H]⁺ 737.4022 (calcd 737.4021), NMR (¹H; ¹³C).

### Nonanoyl-(S)-β³-HPhe-(R)-Trp-(S)-β³-HLys-(R)-4-amino-(R)-5-methyl-dihydro-furan-2-one (Compound 12)

7 mg, >%99 purity, Rt=7.83, HRMS [M+H]⁺ 745.4648 (calcd 745.4647).

### Adamantoyl-(S)-β³-HPhe-(R)-Trp-(S)-β³-HLys-(R)-4-amino-(R)-5-methyl-dihydro-furan-2-one (Compound 13)

30 mg, >99% purity, Rt=7.34, HRMS [M+H]⁺ 767.4496 (calcd 767.4491), NMR (¹H; ¹³C).

### Benzoyl-(S)-β³-HPhe-(R)-Trp-(S)-β³-HLys-(R), (R)—HThr-NH₂ (Compound 14)

15 mg, >93% purity, Rt=6.04, HRMS [M+H]⁺ 726.3973 (calcd 726.3974), NMR (¹H; ¹³C).

### Dihydrocinnemoyl-(S)-β³-HPhe-(R)-Trp-(S)-β³-HLys-(R), (R)—HThr-NH₂ (Compound 15)

14 mg, >97% purity, Rt=6.33, HRMS [M+H]⁺ 754.4289 (calcd 754.4287), NMR (¹H; ¹³C).

### Nonanoyl-(S)-β³-HPhe-(R)-Trp-(S)-β³-HLys-(R), (R)—HThr-NH₂ (Compound 17)

10.5 mg, >97% purity, Rt=7.47, HRMS [M+H]⁺ 762.4909 (calcd 762.4913), NMR (¹H; ¹³C).

### Adamantoyl-(S)-β³-HPhe-(R)-Trp-(S)-β³-HLys-(R), (R)—HThr-NH₂ (Compound 18)

22.5 mg, >87% purity, Rt=7.00, HRMS [M+H]⁺ 784.4762 (calcd 784.4756), NMR (¹H; ¹³C).

### Dihydrocinnemoyl-(S)-β³-HPhe-(R)-1-Me-Trp-(S)-β³-HLys-(R)-4-amino-(R)-5-methyl-dihydro-furan-2-one (Compound 19)

17 mg, 96% purity, Rt=7.12, HRMS [M+H]⁺ 751.41829 (calcd 751.41831), NMR (¹H; ¹³C).

### Adamantoyl-(S)-β³-HPhe-(R)-1-Me-Trp-(S)-β³-HLys-(R)-4-amino-(R)-5-methyl-dihydro-furan-2-one (Compound 20)

21 mg, >99% purity, Rt=7.73, HRMS [M+H]⁺ 781.46524 (calcd 781.46525), NMR (¹H; ¹³C).

### Ac—(S)-β³-HPhe-(R)-1-Me-Trp-(S)-β³-HLys-(R)-4-amino-(R)-5-methyl-dihydro-furan-2-one (Compound 31)

10 mg, >99% purity, Rt=6.02, HRMS [M+H]⁺ 661.37143 (calcd 661.37136), NMR (¹H; ¹³C).

### Ac—(S)-β³-HPhe-(R)-Trp-(S)-β³-HLys-(R)-β³-Leu-diethyl-amide (Compound 22)

9.6 mg, >94% purity, Rt 6.44, HRMS [M+H]⁺ 718.46574 (calcd 718.46559).

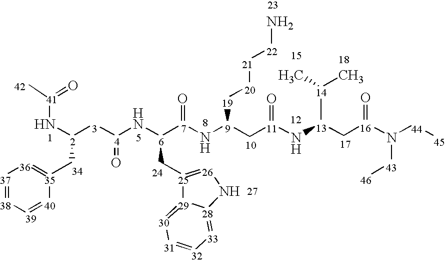

¹H (500 MHz, DMSO, mixture of rotamers): δ (ppm)=0.8 (d, 6H, C₁₈H3, C₁₅H3); 0.98 (t, 3H, C₄₅H3); 0.9-1.09 (m, 2H, C₂₀H2); 1.09 (m, 3H, C₄₆H3); 1.2 (m, 2H, C₁₉H2); 1.30 (m, 2H, C₂₁H2); 1.76 (m, 1H, C₁₄H); 1.69 (s, 3H, C₄₂H3); 2.03, 2.3 (m, 2H, C₁₇H2); 2.10-2.29 (m, 4H, C₃H2, C₁₀H2); 2.52, 2.62 (m, 2H, C₃₄H2); 2.54 (m, 2H, C₂₂H2); 2.9, 3.09 (m, 2H, C₂₄H2); 3.2 (m, 2H, C₄₄H2); 3.3 (m, 2H, C₄₃H2); 3.93 (m, 1H, C₉H); 4.01 (m, 1H, C₁₃H); 4.2 (m, 1H, C₂H); 4.5 (m, 1H, C₆H); 6.98 (t, 1H, C₃₁H); 7.05 (d, 1H, C₃₂H); 7.12 (m, 1H, C₂₆H); 7.15 (m, 1H, C₃₈H); 7.18 (m, 2H, C₃₆H, C₄₀H); 7.23 (m, 2H, C₃₇H, C₃₉H); 7.3 (m, 1H, C₃₃H); 7.58 (m, 1H, C₃₀H); 7.63 (br s, 3H, N₂₃H3⁺); 7.70 (m, 1H, N₁H); 7.72 (m, N₁₂H); 7.8 (d, 1H, N₈H); 8.1 (d, 1H, N₅H); 10.8 (s, N₂₇H).

¹³C (125 MHz; DMSO, mixture of rotamers): δ (ppm)=14 (C₄₅H3); 15.1 (C₄₆H3); 18.4, 20 (C₁₅H3, C₁₈H3); 22.4 (C₂₀H2); 22.5 (C₄₂H3); 27.2 (C₂₁H2); 28.8 (C₂₄H2); 30.2 (C₁₄H); 33.4 (C₁₉H2); 34.7 (C₁₇H2); 38.5 (C₂₂H2); 39.2 (C₄₄H2); 39.5 (C₃₄H2); 40.2 (C₃H2); 41.3 (C₁₀H2); 41.8 (C₄₃H2); 46.3 (C₉H); 48.3 (C₂H); 51.3 (C₁₃H); 54.2 (C₆H); 110.6 (C₂₅); 111.7 (C₃₃H); 118.6 (C₃₁H); 118.8 (C₃₀H); 121.3 (C₃₂H); 123.8 (C₂₆H); 126.4 (C₃₈H); 127.7 (C₂₉); 128.5 (C₃₇H, C₃₉H); 129.6 (C₃₆H, C₄₀H); 136.5 (C₂₈); 139.3 (C₃₅); 167.4; 168.9; 169.6; 169.8; 170.4; 171.4.

### Dihydrocinnemoyl-(S)-β³-HPhe-(R)-Trp-(S)-β³-HLys-(R)-β³-Leu-methyl-phenethyl-amide (Compound 23)

14 mg, >93% purity, Rt=8.09, HRMS [M+H]⁺ 870.5292 (calcd 870.5282).

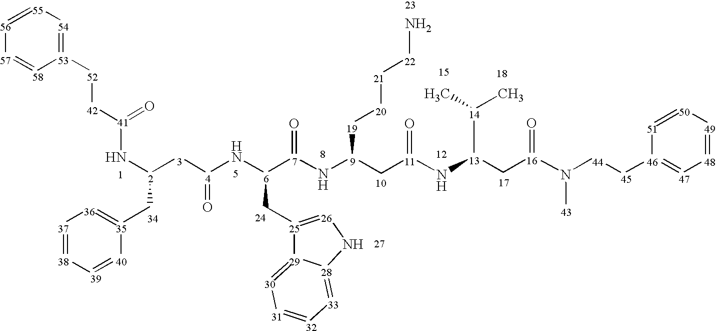

¹H (500 MHz, DMSO, mixture of rotamers): δ (ppm)=0.7 (d, 3H, C₁₈H3); 0.78 (m, 3H, C₁₅H3); 0.89-1.02 (m, 2H, C₂₀H2); 1.15 (m, 2H, C₁₉H2); 1.30 (m, 2H, C₂₁H2); 1.7 (m, 1H, C₁₄H); 2.03, 2.17 (m, 2H, C₁₇H2); 2.10-2.24 (m, 4H, C₃H2, C₁₀H2); 2.25 (m, 2H, C₄₅H2); 2.30 (m, 2H, C₄₂H2); 2.52, 2.62 (m, 2H, C₃₄H2); 2.54 (m, 2H, C₂₂H2); 2.68 (m, 2H, C₅₂H2); 2.78, 2.9 (s, 3H, C₄₃H3); 2.9, 3.09 (m, 2H, C₂₄H2); 3.4, 3.55 (m, 2H, C₄₄H2); 3.9 (m, 1H. C₉H); 3.99 (m, 1H, C₁₃H); 4.2 (m, 1H, C₂H); 4.5 (m, 1H, C₆H); 6.9 (t, 1H, C₃₁H); 7.02 (d, 1H, C₃₂H); 7.12 (m, 1H, C₂₆H); 7.15 (m, 3H, C₃₈H, C₄₉H, C₅₆H); 7.18 (m, 6H, C₃₆H, C₄₀H, C₄₇H, C₅₁H, C₅₄H, C₅₈H); 7.23 (m, 6H, C₃₇H, C₃₉H, C₄₈H, C₅₀H, C₅₅H, C₅₇H); 7.3 (m, 1H, C₃₃H); 7.55 (d, N₁₂H); 7.58 (m, 1H, C₃₀H); 7.62 (br s, 3H, N₂₃H3⁺); 7.72 (m, 1H, N₁H); 7.8 (d, 1H, N₈H); 8.1 (d, 1H, N₅H); 10.8 (s, N₂₇H). ¹³C (125 MHz; DMSO, mixture of rotamers): δ (ppm)=18.2, 19.8 (C₁₅H3), 18.4, 19.6 (C₁₈H3), 22.4 (C₂₀H2), 27.1 (C₁₉H2), 28.5 (C₂₄H2), 31.2 (C₁₄H), 31.5 (C₅₂H2), 33.4 (C₂₁H2), 34.5, 35.7 (C₄₃H3), 34.7 (C₁₇H2), 35.9 (C₄₂H2), 37.5 (C₄₅H2), 39.0 (C₂₂H2), 39.1 (C₃₄H2), 40.2 (C₃H2), 41.3 (C₁₀H2), 46.3 (C₉H), 47.8 (C₂H), 48.3, 51.1, 51.2 (C₄₄H2), 50.9 (C₁₃H), 54.2 (C₆H), 110.6 (C₂₅), 111.7 (C₃₃H), 118.6 (C₃₁H), 118.8 (C₃₀H), 121.3 (C₃₂H), 123.8 (C₂₆H), 126.2-126.8 (C₃₈H, C₄₉H, C₅₆H), 127.7 (C₂₉), 128.4-128.7 (C₃₇H, C₃₉H, C₄₈H, C₅₀H, C₅₅H, C₅₇H), 129.7 (C₃₆H, C₄₀H, C₄₇H, C₅₁H, C₅₄H, C₅₈H), 136.5 (C₂₈), 139.0-139.7 (C₃₅, C₄₆, C₅₃), 169.7, 169.8, 170.3, 170.4 (C₁₁), 171.0, 171.1 (C₇), 171.3, 172.8.

### Dihydrocinnemoyl-(S)-β³-HPhe-(R)-Trp-(S)-β³-HLys-(R)-β³-Leu-diethyl-amide (Compound 24)

8 mg, >99% purity, Rt=7.51, HRMS [M+H]⁺ 808.51248 (calcd 808.51254).

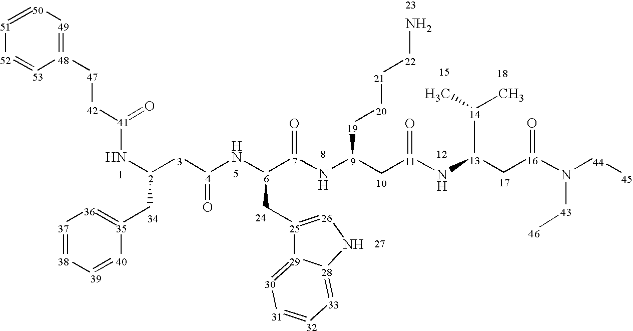

¹H (500 MHz, DMSO, mixture of rotamers): δ (ppm)=0.8 (d, 6H, C₁₈H3, C₁₅H3); 0.98 (t, 3H, C₄₅H3); 0.9-1.06 (m, 2H, C₂₀H2); 1.09 (m, 3H, C₄₆H3); 1.24 (m, 2H, C₁₉H2); 1.37 (m, 2H, C₂₁H2); 1.75 (m, 1H, C₁₄H); 2.10-2.29 (m, 4H, C₃H2, C₁₀H2); 2.23 (t, 2H, C₄₂H2); 2.32 (m, 2H, C₁₇H2); 2.52, 2.62 (m, 2H, C₃₄H2); 2.54 (m, 2H, C₂₂H2); 2.69 (t, 2H, C₄₇H2); 2.9, 3.09 (m, 2H, C₂₄H2); 3.2 (m, 2H, C₄₄H2); 3.21, 3.28 (m, 2H, C₄₃H2); 3.93 (m, 1H, C₉H); 4.0 (m, 1H, C₁₃H); 4.21 (m, 1H, C₂H); 4.5 (m, 1H, C₆H); 6.98 (t, 1H, C₃₁H); 7.05 (d, 1H, C₃₂H); 7.13 (m, 1H, C₂₆H); 7.15 (m, 2H, C₃₈H, C₅₁H); 7.10-7.20 (m, 4H, C₃₆H, C₄₀H, C₄₉H, C₅₃H); 7.21-7.27 (m, 4H, C₃₇H, C₃₉H, C₅₀H, C₅₂H); 7.32 (m, 1H, C₃₃H); 7.58 (m, 1H, C₃₀H); 7.63 (br s, 3H, N₂₃H3⁺); 7.70 (m, 1H, N₁H); 7.72 (m, N₁₂H); 7.8 (d, 1H, N₈H); 8.1 (d, 1H, N₅H); 10.8 (s, N₂₇H).

¹³C (125 MHz; DMSO, mixture of rotamers): δ (ppm)=13.4 (C₄₅H3); 14.7 (C₄₆H3); 18.4, 19.8 (C₁₅H3, C₁₈H3); 22.4 (C₂₀H2); 27.2 (C₂₁H2); 28.8 (C₂₄H2); 30.2 (C₁₄H); 31.5 (C₄₇H2); 32.9 (C₁₉H2); 35.3 (C₁₇H2); 37.5 (C₄₂H2); 38.5 (C₂₂H2); 39.2 (C₄₄H2); 39.5 (C₃₄H2); 40.2 (C₃H2); 41.3 (C₁₀H2); 41.8 (C₄₃H2); 46.3 (C₉H); 48.3 (C₂H); 51.3 (C₁₃H); 54.2 (C₆H); 110.6 (C₂₅); 111.7 (C₃₃H); 118.6 (C₃₁H); 118.8 (C₃₀H); 121.3 (C₃₂H); 123.8 (C₂₆H); 126.3 (C₃₈H, C₅₁H); 127.7 (C₂₉); 128.5 (C₃₇H, C₃₉H, C₅₀H, C₅₂H); 129.6 (C₃₆H, C₄₀H, C₄₉H, C₅₃H); 136.5 (C₂₈); 139.2 (C₃₅, C₄₈); 167.4; 169.6; 169.8; 170.3; 171.0; 171.3.

### Ac—(S)-β³-HPhe-(R)-Trp-(S)-β³-HLys-(R)-β³-Leu-methyl-phenethyl-amide (Compound 25)

14 mg, >99% purity, Rt=7.19, HRMS [M+H]⁺ 780.48124 (calcd 780.48124), NMR (¹H; ¹³C).

### Cyclohexanoyl-(S)-β³-HPhe-(R)-Trp-(S)-β³-HLys-(R), (R)—HThr-NH₂ (Compound 26)

13.7 mg, >89% purity, Rt=6.33, HRMS [M+H]⁺ 732.44501 (calcd 732.44485), NMR (¹H; ¹³C).

### Adamantoyl-(S)-β³-HPhe-(R)-Trp-(S)-β³-HLys-(R)-β³-Leu-methyl-phenethyl-amide (Compound 27)

10 mg, >80% purity, Rt=8.65, HRMS [M+H]⁺ 900.5751 (calcd 900.5751).

### (S)-β³-HPhe-(R)-Trp-(S)-β³-HLys-(R)-4-amino-(R)-5-methyl-dihydro-furan-2-one (Compound 28)

35 mg, >99% purity, Rt=4.95 HRMS [M+H]⁺ 605.34524 (calcd 605.34514).

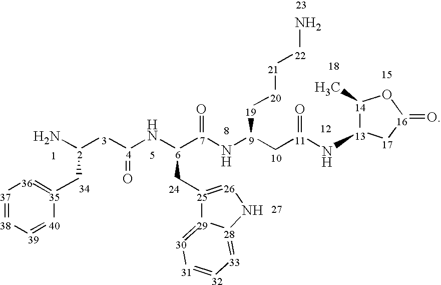

¹H (500 MHz, DMSO): δ (ppm)=1.02 (m, 2H, C₂₀H2); 1.1 (d, 3H, C₁₈H3); 1.29 (m, 2H, C₁₉H2); 1.4 (m, 2H, C₂₁H2); 2.13 (m, 2H, C₁₀H2); 2.17, 2.95 (m, 2H, C₁₇H2); 2.23, 2.32 (m, 2H, C₃H2); 2.51 (m, 2H, C₂₂H2); 2.7, 2.86 (m, 2H, C₃₄H2); 2.98, 3.1 (m, 2H, C₂₄H2); 3.53 (m, 1H, C₂H); 4.02 (m, 1H, C₉H); 4.48 (m, 1H, C₁₃H); 4.58 (m, 1H, C₆H); 4.67 (m, 1H, C₁₄H); 6.98 (t, 1H, C₃₂H); 7.06 (d, 1H, C₃₁H); 7.11 (m, 2H, C₃₆H, C₄₀H); 7.14 (m, 1H, C₂₆H); 7.25 (m, 1H, C₃₈H); 7.29 (m, 2H, C₃₇H, C₃₉H); 7.32 (m, 1H, C₃₃H); 7.65 (m, 1H, C₃₀H); 7.78 (br s, 3H, N₂₃H3⁺); 7.9 (br s, 3H, N₁H3⁺); 7.99 (d, 1H, N₈H); 8.35 (m, 1H, N₁₂H); 8.5 (m, 1H, N₅H); 10.88 (brs, N₂₇H).

¹³C (125 MHz; DMSO): δ (ppm)=15 (C₁₈H3); 22.4 (C₂₀H2); 27.2 (C₂₁H2); 28.7 (C₂₄H2); 33.5 (C₁₉H2); 35.6 (C₁₇H2); 36.0 (C₃H2); 38.4 (C₃₄H2); 39.1 (C₂₂H2); 41 (C₁₀H2); 46.3 (C₉H); 48.7 (C₁₃H); 49.8 (C₂H); 54.1 (C₆H); 79.2 (C₁₄H); 110.3 (C₂₅); 111.7 (C₃₃H); 118.6 (C₃₂H); 118.9 (C₃₀H); 121.3 (C₃₁H); 124.1 (C₂₆H); 127.4 (C₃₈H); 127.7 (C₂₉); 129.1 (C₃₇H, C₃₉H); 129.8 (C₃₆H, C₄₀H); 136.5 (C₂₈, C₃₅); 169.7 (C₄), 170.5 (C₁₁); 171.1 (C₇); 175.7 (C₁₆).

### Ac—(S)-β³-HPhe-(R)-Trp-(S)-β³-HLys-cyclopentyl-amide (Compound 31)

39.1 mg, >99% purity, Rt=6.23, MS [M+H]⁺ 617.7, HRMS [M+H]⁺ 617.3809 (calcd 617.3810), [M+Na]⁺ 639.3630 (calcd 639.3629).

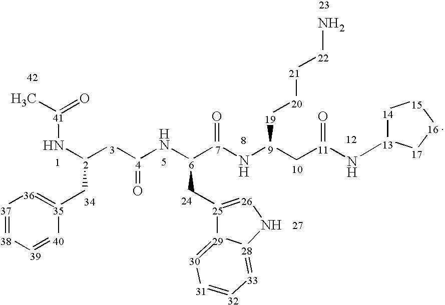

¹H (500 MHz, DMSO): δ (ppm)=1.02 (m, 2H, C₂₀H2); 1.18-1.23 (m, 2H, C₁₉H2); 1.24-1.33, 1.43-1.52 (m, 4H, C₁₅H2, C₁₆H2); 1.34-1.42 (m, 2H, C₂₁H2); 1.53-1.63, 1.70-1.8 (m, 4H, C₁₄H2, C₁₇H2); 1.7 (m, 3H, C₄₂H3); 2.09-2.35 (m, 4H, C₃H2, C₁₀H2); 2.55-2.78 (m, 4H, C₃₄H2, C₂₂H2); 2.9, 3.09 (m, 2H, C₂₄H2); 3.9-4.05 (m, 2H, C₁₃H, C₉H); 4.2 (m, 1H, C₂H); 4.5 (m, 1H, C₆H); 6.99 (m, 1H, C32H); 7.08 (m, 1H, C31H); 7.13 (m, 2H, C₃₆H, C₄₀H); 7.13 (m, 1H, C₂₆H); 7.22-7.28 (m, 2H, C₃₇H, C₃₉H); 7.25 (m, 1H, C₃₈H); 7.32 (m, 1H, C₃₃H); 7.59 (m, 1H, C₃₀H); 7.72 (br s, 3H, N₂₃H3⁺); 7.74 (m, 1H, N₁H); 7.78 (m, 1H, N₁₂H); 7.82 (d, 1H, N₈H); 8.1 (d, 1H, N₅H); 10.85 (s, N₂₇H).

¹³C (125 MHz; DMSO): δ (ppm)=22.4 (C₂₀H2); 23.0 (C₄₂H3); 23.7 (C₁₅H2, C₁₆H2); 27.0 (C₂₁H2); 28.3 (C₂₄H2); 32.5, 32.6 (C₁₄H2, C₁₇H2); 33.1 (C₁₉H2); 39.2 (C₂₂H2); 40 (C₃₄H2); 40.5 (C₁₀H2); 41 (C₃H2); 46.2 (C₉H); 48.3 (C₂H); 50.5 (C₁₃H); 54.2 (C₆H); 110.5 (C₂₅); 111.6 (C₃₃H); 118.5 (C32H); 118.7 (C₃₀H); 121.2 (C31H); 123.7 (C₂₆H); 126.3 (C₃₈H); 127.6 (C₂₉); 128.4 (C₃₇H, C₃₉H); 129.5 (C₃₆H, C₄₀H); 136.4 (C₂₈); 139.2 (C₃₅); 168.8; 169.7; 170.3; 171.3.

### Dihydrocinnemoyl-(S)-β³-HPhe-(R)-Trp-(S)-β³-HLys-cyclopentyl-amide (Compound 32)

13.2 mg, >96% purity, Rt=7.28, MS [M+H]⁺ 707.7, HRMS [M+H]⁺ 707.4281 (calcd 707.4279), [M+Na]⁺ 729.4099 (calcd 729.4099).

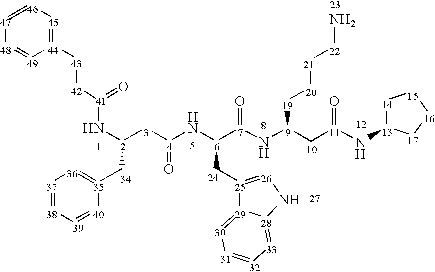

¹H (500 MHz, DMSO): δ (ppm)=1.1 (m, 2H, C₂₀H2); 1.28-1.33 (m, 2H, C₁₉H2); 1.34-1.44, 1.48-1.59 (m, 4H, C₁₅H2, C₁₆H2); 1.42-1.47 (m, 2H, C₂₁H2); 1.6-1.72, 1.73-1.9 (m, 4H, C₁₄H2, C₁₇H2); 2.11-2.45 (m, 6H, C₃H2, C₁₀H2, C₄₂H2); 2.65-2.85 (m, 6H, C₃₄H2, C₂₂H2, C₄₃H2); 2.97, 3.13 (m, 2H, C₂₄H2); 3.95-4.1 (m, 2H, C₁₃H, C₉H); 4.3 (m, 1H, C₂H); 4.57 (m, 1H, C₆H); 7.05 (m, 1H, C32H); 7.15 (m, 1H, C31H); 7.17-7.28 (m, 7H, C₃₆H, C₄₀H, C₄₅H, C₄₉H, C₃₈H, C₄₇H, C₂₆H); 7.28-7.32 (m, 4H, C₃₇H, C₃₉H, C₄₆H, C₄₈H); 7.39 (m, 1H, C₃₃H); 7.64 (m, 1H, C₃₀H); 7.31 (br s, 3H, N₂₃H3⁺); 7.79 (m, 1H, N₁H); 7.82 (m, 1H, N₁₂H); 7.91 (d, 1H, N₈H); 8.17 (d, 1H, N₅H); 10.9 (s, N₂₇H).

¹³C (125 MHz; DMSO): δ (ppm)=22.4 (C₂₀H2); 23.7 (C₁₅H2, C₁₆H2); 27.0 (C₂₁H2); 28.3 (C₂₄H2); 31.4 (C₄₃H2); 32.5, 32.6 (C₁₄H2, C₁₇H2); 33.1 (C₁₉H2); 37.5 (C₄₂H2); 39.2 (C₂₂H2); 40 (C₃₄H2); 40.5 (C₁₀H2); 41 (C₃H2); 46.2 (C₉H); 48.3 (C₂H); 50.5 (C₁₃H); 54.2 (C₆H); 110.5 (C₂₅); 111.6 (C₃₃H); 118.5 (C32H); 118.7 (C₃₀H); 121.2 (C31H); 123.7 (C₂₆H); 126.2 (C₃₈H, C₄₇H); 127.6 (C₂₉); 128.4 (C₃₇H, C₃₉H, C₄₆H, C₄₈H); 129.5 (C₃₆H, C₄₀H, C₄₅H, C₄₉H); 136.4 (C₂₈); 139.1 (C₄₄, C₃₅); 168.8; 169.7; 170.3; 171.3.

### Adamantoyl-(S)-β³-HPhe-(R)-Trp-(S)-β³-HLys-cyclopentyl-amide (Compound 34)

23.4 mg, >99% purity, Rt=7.88, MS [M+H]⁺ 737.6, HRMS [M+H]⁺ 737.4750 (calcd 737.4749), [M+Na]⁺ 759.4569 (calcd 759.4568).

### (S)-β³-HPhe-(R)-Trp-(S)-β³-HLys-(R), (R)—HThr-NH₂ (Compound 36)

14 mg, >99% purity, Rt=4.82, MS [M+H]⁺ 622.6, [M+2H]²⁺ 312.0.

### Ac—(S)-β³-HPhe-(R)—N-Me-1-Me-Trp-(S)-β³-HLys-(R)-4-amino-(R)-5-methyl-dihydro-furan-2-one (Compound 37)

29.8 mg, >99% purity, Rt=6.05 (determined with System I), Rt=4.95 (determined with System II), MS [M+H]⁺ 675.8, HRMS [M+H]⁺ 675.3862 (calcd 675.3862), [M+Na]⁺ 697.3683 (calcd 697.3684).

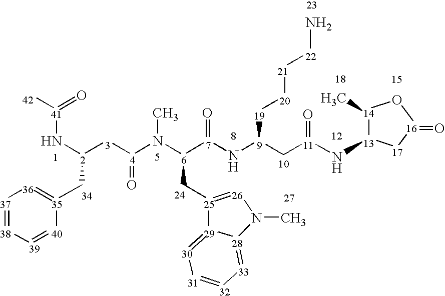

¹H (500 MHz, DMSO, mixture of rotamers, T=300K): δ (ppm)=1.12 (d, 3H, C₁₈H3); 1.19 (m, 2H, C₂₀H2); 1.39 (m, 2H, C₁₉H2); 1.48 (m, 2H, C₂₁H2); 1.66, 1.69 (m, 3H, C₄₂H3); 1.86, 2.3, 2.37 (m, 2H, C₃H2); 2.04, 2.27, 2.57, 2.68 (m, 2H, C₃₄H2); 2.13-2.31 (m, 2H, C₁₀H2); 2.24, 2.94 (m, 2H, C₁₇H2); 2.66 (m, 2H, C₂₂H2); 2.81, 2.87 (m, 3H, CH3-N₅); 2.91, 3.01, 3.28 (m, 2H, C₂₄H2); 3.66, 3.69 (d, 3H, CH3-N₂₇); 4.09, 4.18 (m, 1H, C₂H); 4.12 (m, 1H, C₉H); 4.49 (m, 1H, C₁₃H); 4.50, 4.68 (m, 1H, C₁₄H); 4.59, 5.29 (m, 1H, C₆H); 7.0 (m, 2H, C₃₂H, C₂₆H); 7.0-7.08 (m, 2H, C₃₆H, C₄₀H); 7.11 (m, 1H, C₃₁H); 7.15 (m, 1H, C₃₈H); 7.30 (m, 2H, C₃₇H, C₃₉H); 7.33, 7.39 (m, 1H, C₃₃H); 7.6 (m, 1H, C₃₀H); 7.65 (d, 1H, N₈H); 7.7 (br s, 3H, N₂₃H3⁺); 7.84, 7.95 (d, 1H, N₁H); 8.38, 8.4 (m, 1H, N₁₂H).

### N-Me-(S)-β³-HPhe-(R)-Trp-(S)-β³-HLys-(R)-4-amino-(R)-5-methyl-dihydro-furan-2-one (Compound 38)

66 mg, >99% purity, Rt=4.96 (determined with System I), Rt=3.71 (determined with System II), MS [M+H]⁺ 619.8, [M+2H]²⁺ 310.5, HRMS [M+H]⁺ 619.3601 (calcd 619.3603), [M+Na]⁺ 641.3422 (calcd 641.3422).

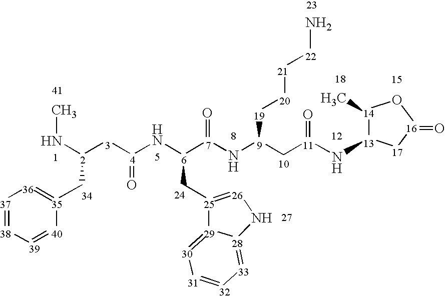

¹H (500 MHz, DMSO): δ (ppm)=1.02 (m, 2H, C₂₀H2); 1.1 (d, 3H, C₁₈H3); 1.29 (m, 2H, C₁₉H2); 1.4 (m, 2H, C₂₁H2); 2.13 (m, 2H, C₁₀H2); 2.29, 2.95 (m, 2H, C₁₇H2); 2.40 (m, 2H, C₃₄H2); 2.47 (t, 3H, C₄₁H3); 2.62 (m, 2H, C₂₂H2); 2.61, 2.96 (m, 2H, C₃H2); 2.98, 3.09 (m, 2H, C₂₄H2); 3.53 (m, 1H, C₂H); 4.02 (m, 1H, C₉H); 4.48 (m, 1H, C₁₃H); 4.58 (m, 1H, C₆H); 4.67 (m, 1H, C₁₄H); 6.98 (t, 1H, C₃₂H); 7.06 (d, 1H, C₃₁H); 7.11 (m, 2H, C₃₆H, C₄₀H); 7.14 (m, 1H, C₂₆H); 7.25 (m, 1H, C₃₈H); 7.29 (m, 2H, C₃₇H, C₃₉H); 7.32 (m, 1H, C₃₃H); 7.65 (m, 1H, C₃₀H); 7.78 (br s, 3H, N₂₃H3⁺); 7.99 (d, 1H, N₈H); 8.35 (m, 1H, N₁₂H); 8.5 (m, 1H, N₅H); 8.59 (brs, 2H, N₁H2⁺); 10.88 (brs, N₂₇H).

¹³C (125 MHz; DMSO): δ (ppm)=15 (C₁₈H3); 22.5 (C₂₀H2); 27.2 (C₂₁H2); 28.8 (C₂₄H2); 30.7 (C₄₁H3); 33.5 (C₁₉H2); 33.7 (C₃H2); 35.6 (C₁₇H2); 36.4 (C₃₄H2); 39.1 (C₂₂H2); 41 (C₁₀H2); 46.3 (C₉H); 48.7 (C₁₃H); 56.0 (C₆H); 57.2 (C₂H); 79.2 (C₁₄H); 110.3 (C₂₅); 111.7 (C₃₃H); 118.6 (C₃₂H); 119 (C₃₀H); 121.3 (C₃₁H); 124.1 (C₂₆H); 127.4 (C₃₈H); 127.7 (C₂₉); 129.1 (C₃₇H, C₃₉H); 129.8 (C₃₆H, C₄₀H); 136.5 (C₂₈); 136.6 (C₃₅); 169.6 (C₄), 170.5 (C₁₁); 171.0 (C₇); 175.7 (C₁₆).

### Ac—N-Me-(S)-β³-HPhe-(R)-Trp-(S)-β³-HLys-(R)-4-amino-(R)-5-methyl-dihydro-furan-2-one (Compound 39)

27 mg, >99% purity, Rt=5.67 (determined with System I), Rt=4.48 (determined with System II), MS [M+H]⁺ 661.7, HRMS [M+H]⁺ 661.3708 (calcd 661.3708), [M+Na]⁺ 683.3529 (calcd 683.3528).

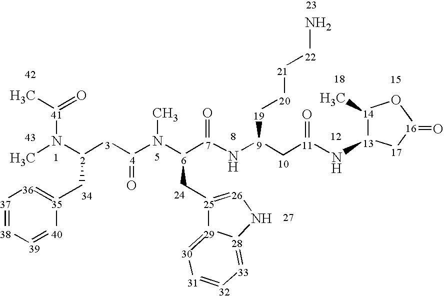

¹H (500 MHz, DMSO, mixture of rotamers, T=300K): δ (ppm)=0.95 (m, 2H, C₂₀H2); 1.12 (d, 3H, C₁₈H3); 1.22 (m, 2H, C₁₉H2); 1.35 (m, 2H, C₂₁H2); 1.53, 1.6 (m, 3H, C₄₂H3); 2.17 (m, 2H, C₁₀H2); 2.23, 2.93 (m, 2H, C₁₇H2); 2.31-2.39 (m, 2H, C₃H2); 2.5-2.69 (m, 2H, C₃₄H2); 2.52, 2.62 (m, 3H, C₄₃H3); 2.72 (m, 2H, C₂₂H2); 2.83, 3.05 (m, 2H, C₂₄H2); 3.93, 4.02 (m, 1H, C₉H); 4.29 (m, 1H, C₂H); 4.45 (m, 1H, C₆H); 4.49 (m, 1H, C₁₃H); 4.68 (m, 1H, C₁₄H); 6.98 (t, 1H, C₃₂H); 7.05 (m, 1H, C₃₁H); 7.06 (m, 1H, C₂₆H); 7.13 (m, 2H, C₃₆H, C₄₀H); 7.19 (m, 1H, C₃₈H); 7.28 (m, 2H, C₃₇H, C₃₉H); 7.31 (m, 1H, C₃₃H); 7.53 (m, 1H, C₃₀H); 7.62 (br s, 3H, N₂₃H3⁺); 7.77, 7.86 (d, 1H, N₈H); 8.31 (m, 1H, N₅H); 8.09, 8.36 (m, 1H, N₁₂H); 10.78, 10.82 (s, N₂₇H).

### Ac—(S)-β³-HPhe-(R)—N-Me-Trp-(S)-β³-HLys-(R)-4-amino-(R)-5-methyl-dihydro-furan-2-one (Compound 40)

12 mg, >99% purity, Rt=5.67 (determined with System I), Rt=4.53 (determined with System II), MS [M+H]⁺ 661.8, HRMS [M+H]⁺ 661.3709 (calcd 661.3708), [M+Na]⁺ 683.3530 (calcd 683.3528).

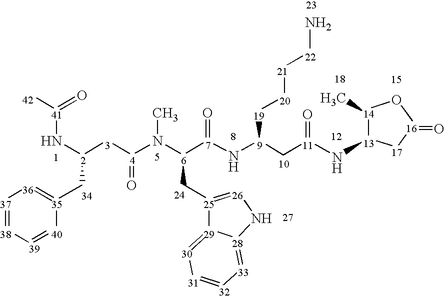

¹H (500 MHz, DMSO, mixture of rotamers): δ (ppm)=1.0 (d, 3H, C₁₈H3); 1.05 (m, 2H, C₂₀H2); 1.29 (m, 2H, C₁₉H2); 1.38 (m, 2H, C₂₁H2); 1.53, 1.6 (m, 3H, C₄₂H3); 1.7, 2.27 (m, 2H, C₃H2); 1.96, 2.19, 2.49 (m, 2H, C₃₄H2); 2.17 (m, 2H, C₁₀H2); 2.59 (m, 2H, C₂₂H2); 2.7, 2.73 (m, 3H, CH3-N₅); 2.19, 2.82 (m, 2H, C₁₇H2); 2.82, 3.17 (m, 2H, C₂₄H2); 4.0 (m, 1H, C₉H); 4.08 (m, 1H, C₂H); 4.39 (m, 1H, C₁₃H); 4.55 (m, 1H, C₁₄H); 5.19 (m, 1H, C₆H); 6.86 (t, 1H, C₃₁H); 6.9 (d, 1H, C₃₂H); 6.95 (m, 1H, C₂₆H); 6.9-6.98 (m, 2H, C₃₆H, C₄₀H); 7.05 (m, 1H, C₃₈H); 7.12 (m, 2H, C₃₇H, C₃₉H); 7.2 (m, 1H, C₃₃H); 7.48 (m, 1H, C₃₀H); 7.5 (m, 1H, N₈H); 7.52 (br s, 3H, N₂₃H3⁺); 7.7, 7.8 (d, 1H, N₁H); 8.2 (m, 1H, N₁₂H); 10.62, 10.72 (s, N₂₇H).

¹³C (125 MHz; DMSO, mixture of rotamers): δ (ppm)=15 (C₁₈H3); 22.4 (C₂₀H2); 23 (C₄₂H3); 24.5, 25.2 (C₂₄H2); 27.2 (C₂₁H2); 29.1 31.3 (CH3N₅); 33.3 (C₁₉H2); 35.6 (C₁₇H2); 38.2, 38.9 (C₃H2); 39.2 (C₂₂H2); 39, 40, 41.2 (C₃₄H2); 41 (C₁₀H2); 46.1 (C₉H); 48 (C₂H); 48.7 (C₁₃H); 56.4, 60.2 (C₆H); 79.2 (C₁₄H); 110.6 (C₂₅); 111.7 (C₃₃H); 118.6 (C₃₁H); 118.8 (C₃₀H); 121.3 (C₃₂H); 123.8 (C₂₆H); 126.4 (C₃₈H); 127.7 (C₂₉); 128.4 (C₃₇H, C₃₉H); 129.7 (C₃₆H, C₄₀H); 136.5 (C₂₈); 139.3 (C₃₅); 170.4 (C₄), 170.6 (C₁₁); 171.3 (C₇); 174.8 (C₄₁); 175.7 (C₁₆).

### Butyroyl-(S)-β³-HPhe-(R)-Trp-(S)-β³-HLys-methyl-pentyl-amide (Compound 41)

44.6 mg, >99% purity, Rt=7.15, MS [M+H]⁺ 661.8, HRMS [M+H]⁺ 661.4436 (calcd 661.4436).

### Butyroyl-(S)-β³-HPhe-(R)-Trp-(S)-β³-HLys-methyl-butyl-amide (Compound 42)

38.3 mg, >99% purity, Rt=6.83, MS [M+H]⁺ 647.8, HRMS [M+H]⁺ 647.4280 (calcd 647.4279).

### Propionyl-(S)-β³-HPhe-(R)-Trp-(S)-β³-HLys-methyl-butyl-amide (Compound 43)

35.1 mg, >97% purity, Rt=6.73, MS [M+H]⁺ 633.9, HRMS [M+H]⁺ 633.4123 (calcd 633.4123).

### Ace-(S)-β³-HPhe-(R)-Trp-(S)-β³-HLys-pentyl-amide (Compound 45)

23.5 mg, >99% purity, Rt=6.52, MS [M+H]⁺ 619.7. HRMS [M+H]⁺ 619.3964 (calcd 619.3966), [M+Na]⁺ 641.3786 (calcd 641.3786).

### Propionyl-N-Me-(S)-β³-HPhe-(R)-Trp-(S)-β³-HLys-cyclopentyl-amide (Compound 46)

36.9 mg, >99% purity, Rt=6.52, MS [M+H]⁺ 645.9, HRMS [M+H]⁺ 645.4122 (calcd 645.4123).

### Ace-(S)-β³-HPhe-(R)-Trp-(S)-β³-HLys-methyl-pentyl-amide (Compound 47)

13.8 mg, >99% purity, Rt=6.70, MS [M+H]⁺ 634.0, HRMS [M+H]⁺ 633.4120 (calcd 633.4123), [M+Na]⁺ 655.3942 (calcd 655.3942).

### Propionyl-(S)-β³-HPhe-(R)-Trp-(S)-β³-HLys-methyl-pentyl-amide (Compound 49)

11.9 mg, >99% purity, Rt=6.97, MS [M+H]⁺ 647.2, [M−H]⁻ 645.3, [M+TFA]⁻ 759.2, HRMS [M+H]⁺ 647.4277 (calcd 647.4279).

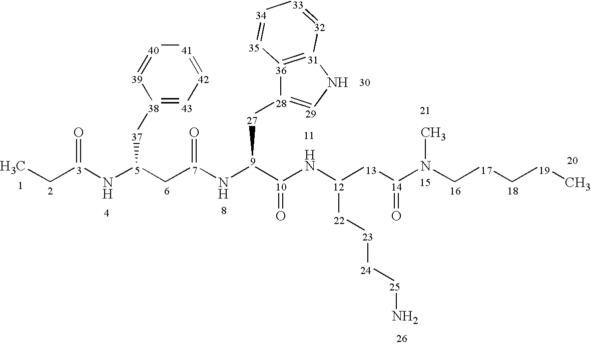

¹H (500 MHz, DMSO, mixture of rotamers, T=393K): δ (ppm)=0.86 (m, 3H, C₁H3); 1.95 (q, 2H, C₂H2); 7.55 (m, 1H, N₄H); 4.2 (m, 1H, C₅H); 2.21 (m, 2H, C₆H2); 8.05 (m, 1H, N₈H); 4.48 (m, 1H, C₉H); 7.7 (d, 1H, N₁₁H); 3.96 (m, 1H, C₁₂H); 2.26, 2.39 (m, 2H, C₁₃H2); 3.12-3.22 (m, 2H, C₁₆H2); 1.41 (m, 2H, C₁₇H2); 1.00 (m, 2H, C₁₈H2); 1.22 (m, 2H, C₁₉H2); 0.82 (m, 3H, C₂₀H3); 2.72, 2.82 (m, 3H, C₂₁H3); 1.24, 1.32 (m, 2H, C₂₂H2); 1.12, 1.22 (m, 2H, C₂₃H2); 1.37 (m, 2H, C₂₄H2); 2.62 (m, 2H, C₂₅H2); 7.6 (br s, 3H, N₂₆H3⁺); 2.89, 3.06 (m, 2H, C₂₇H2); 7.12 (m, 1H, C₂₉H); 10.78 (s, 1H, N₃₀H); 7.32 (m, 1H, C₃₂H); 7.03 (m, 1H, C₃₃H); 6.98 (m, 1H, C₃₄H); 7.6 (d, 1H, C₃₅H); 2.6-2.7 (m, 2H, C₃₇H); 7.1 (m, 2H, C₃₉H, C₄₃H); 7.26 (m, 2H, C₄₀H, C₄₂H); 7.18 (m, C₄₁H).

¹³C (125 MHz; DMSO, mixture of rotamers): δ (ppm)=10.5 (C₁H3), 14.3 (C₂₀H3), 22.3 (C₁₉H2), 22.6 (C₁₈H2), 26.7 (C₂₄H2), 27.1 (C₁₇H2), 28.7 (C₂₇H2), 28.9 (C₂₃H2), 29.1 (C₂H2), 33.1, 35.2 (C₂₁H3), 33.3 (C₂₂H2), 37.9 (C₁₃H2), 38.5 (C₂₅H2), 39.1 (C₃₇H2), 40.8 (C₆H2), 46.1, 46.4 (C₁₂H), 47.0 (C₃₇H2), 48.2 (C₅H), 47.0, 49.4 (C₁₆H2), 54.2 (C₉H), 110.6 (C₂₈), 111.7 (C₃₂H), 118.6 (C₃₄H), 118.8 (C₃₅H), 121.3 (C₃₃H), 123.8 (C₂₉H), 126.4 (C₄₁H), 127.7 (C₃₆), 128.4 (C₄₀H, C₄₂H), 129.6 (C₃₉H, C₄₃H), 136.5 (C₃₁), 139.3 (C₃₈), 170.0 (C₁₄), 170.4 (C₇), 171.4 (C₁₀), 172.6 (C₃).

IR (microscope in transmission, cm⁻¹): v=3282 (NH), 3062, 2934 (CH), 2871, 1643 (4× C═O), 1545 (amid), 1457, 1440, 1378, 1356, 1287, 1235, 1202, 1178, 1134, 1031, 1010, 914, 834, 799, 743, 721, 701.

### Propionyl-(S)-β³-HPhe-(R)-Trp-(S)-β³-HLys-pentyl-amide (Compound 51)

14.3 mg, >99% purity, Rt=6.71, MS [M+H]⁺ 633.4. HRMS [M+H]⁺ 633.4122 (calcd 633.4123), [M+Na]⁺ 655.3943 (calcd 655.3942).

### Ace-(S)-β³-HPhe-(R)-Trp-(S)-β³-HLys-butyl-amide (Compound 52)

30.3 mg, >97% purity, Rt=6.20, MS [M+H]⁺ 605.5, HRMS [M+H]⁺ 605.3810 (calcd 605.3810), [M+Na]⁺ 627.3630 (calcd 627.3629).

### Propionyl-(S)-β³-HPhe-(R)-Trp-(S)-β³-HLys-butyl-amide (Compound 53)

36.5 mg, >99% purity, Rt=6.43, MS [M+H]⁺ 619.5, HRMS [M+H]⁺ 619.3966 (calcd 619.3966), [M+Na]⁺ 641.3787 (calcd 641.3786).

### Butyroyl-(S)-β³-HPhe-(R)-Trp-(S)-β³-HLys-butyl-amide (Compound 54)

38.3 mg, >99% purity, Rt=6.80, MS [M+H]⁺ 633.5, HRMS [M+H]⁺ 633.4126 (calcd 633.4123), [M+Na]⁺ 655.3943 (calcd 655.3942).

### Butyroyl-(S)-β³-HPhe-(R)-Trp-(S)-β³-HLys-pentyl-amide (Compound 55)

19.2 mg, >99% purity, Rt=6.91, MS [M+H]⁺ 647.8. HRMS [M+H]⁺ 647.4278 (calcd 647.4279), [M+Na]⁺ 669.4099 (calcd 669.4099).

### Butyroyl-(S)-β³-HPhe-(R)-Trp-(S)-β³-HLys-cyclopentyl-amide (Compound 56)

24.7 mg, >99% purity, Rt=6.65, MS [M+H]⁺ 644.9. HRMS [M+H]⁺ 645.4123 (calcd 645.4123), [M+Na]⁺ 667.3946 (calcd 667.3942).

### Propionyl-(S)-β³-HPhe-(R)-Trp-(S)-β³-HLys-cyclopentyl-amide (Compound 57)

19.1 mg, >96% purity, Rt=6.4, MS [M+H]⁺ 631.6, HRMS [M+H]⁺ 631.3966 (calcd 631.3966), [M+Na]⁺ 653.3785 (calcd 653.3786).

### Ace-(S)-β³-HPhe-(R)-Trp-(S)-β³-HLys-NH₂ (Compound 58)

28.2 mg, >99% purity, Rt=5.36, MS [M+H]⁺ 548.7, HRMS [M+H]⁺ 549.3182 (calcd 549.3184), [M+Na]⁺ 571.3003 (calcd 571.3003).

### (S)-β³-HPhe-(R)-Trp-(S)-β³-HLys-NH₂ (Compound 59)

26 mg, >99% purity, Rt=4.69, MS [M+H]⁺ 507.5, [M+2H]²⁺ 254.5, HRMS [M+H]⁺ 507.3079 (calcd 507.3078), [M+Na]⁺ 529.2900 (calcd 529.2898).

### Propionyl-(S)-β³-HPhe-(R)-Trp-(S)-β³-HLys-NH₂ (Compound 60)

22 mg, >97% purity, Rt=5.52, MS [M+H]⁺ 563.6, [M+2H]²⁺ 282.5.

### Butyroyl-(S)-β³-HPhe-(R)-Trp-(S)-β³-HLys-NH₂ (Compound 61)

23 mg, >96% purity, Rt=5.77, MS [M+H]⁺ 577.5, [M+2H]²⁺ 289.6, HRMS [M+H]⁺ 577.3496 (calcd 577.3497), [M+Na]⁺ 599.3315 (calcd 599.3316).

### (S)-β³-HPhe-(R)-Trp-(S)-β³-HLys-cyclopentyl-amide (Compound 62)

47 mg, >96% purity, Rt=5.49, MS [M+H]⁺ 575.6, [M+2H]²⁺ 288.6, HRMS [M+H]⁺ 575.3706 (calcd 575.3704), [M+Na]⁺ 597.3527 (calcd 597.3524).

### Propionyl-(S)-β³-HPhe-(R)—N-Me-Trp-(S)-β³-HLys-methyl-pentyl-amide (Compound 63)

36 mg, >99% purity, Rt=7.30, MS [M+H]⁺ 661.2, MS [M−H]⁻ 659.2, [M+TFA]⁻ 773.2, HRMS [M+H]⁺ 661.4435 (calcd 661.4436), [M+Na]⁺ 683.4258 (calcd 683.4255).

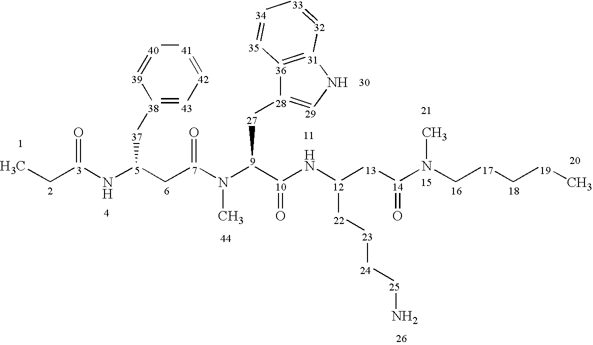

¹H (500 MHz, DMSO, mixture of rotamers; T=393K): δ (ppm)=0.82 (m, 3H, C₂₀H3); 0.86 (m, 3H, C₁H3); 1.18-1.4 (m, 7H, C₁₈H2, C₆H2, C₂₃H2, C₁₉H2); 1.41-1.63 (m, 7H, C₁₇H2, C₂₄H2, C₂₂H2); 1.98 (q, 2H, C₂H2); 2.4, 2.52 (m, 2H, C₁₃H2); 2.76 (m, 2H, C₂₅H2); 2.68-2.8 (m, 2H, C₃₇H2); 2.87 (m, 6H, C₂₁H3, C₄₄H3); 3.0, 3.3 (m, 2H, C₂₇H2); 3.22 (m, 2H, C₁₆H2); 4.12 (m, 1H, C₁₂H); 4.22 (m, 1H, C₅H); 5.2 (m, 1H, C₉H); 6.98 (m, 1H, C₃₄H); 7.03 (m, 1H, C₃₃H); 7.05 (m, 1H, C₂₉H); 7.09 (m, 2H, C₃₉H, C₄₃H); 7.13 (m, C₄₁H); 7.20 (m, 2H, C₄₀H, C₄₂H); 7.30 (m, 1H, C₃₂H); 7.5 (br s, 3H, N₂₆H3⁺); 7.55 (d, 1H, C₃₅H); 10.4 (s, 1H, N₃₀H).

IR (microscope in transmission, cm⁻¹): v=3282 (NH), 3060, 2934 (CH), 2871, 1674, 1647, 1544 (amid), 1493, 1457, 1406, 1341, 1287, 1202, 1177, 1133, 1030, 1010, 921, 833, 799, 744, 721, 702.

### Propionyl-(S)-β³-HPhe-(R)—N-Me-Trp-(S)-β³-HLys-methyl-hexyl-amide (Compound 65)

25 mg, >99% purity, Rt=7.60, MS [M+H]⁺ 675.7, HRMS [M+H]⁺ 675.4593 (calcd 675.4592), [M+Na]⁺ 697.4412 (calcd 697.4412).

### Propionyl-(S)-β³-HPhe-(R)—N-Benzyl-Trp-(S)-β³-HLys-methyl-pentyl-amide (Compound 66)

20 mg, >98% purity, Rt=8.10, MS [M+H]⁺ 737.8, [M+2H]²⁺ 369.8, [M−H]- 735.2, [M+TFA]- 849.2, HRMS [M+H]⁺ 737.4743 (calcd 737.4749), [M+Na]⁺ 759.4569 (calcd 759.4568).

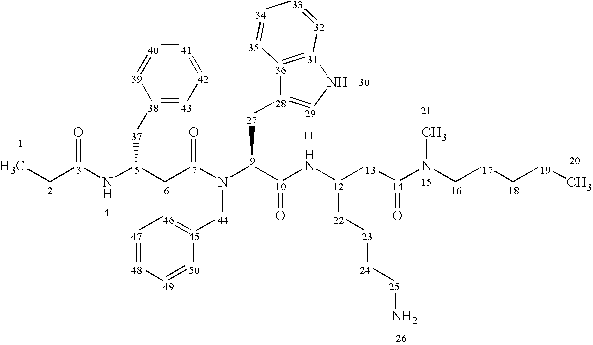

¹H (500 MHz, DMSO, mixture of rotamers; T=393K): δ (ppm)=0.88 (m, 3H, C₂₀H3); 0.93 (m, 3H, C₁H3); 1.09 (m, 2H, C₁₈H2); 1.21 (m, 2H, C₂₃H2); 1.31 (m, 3H, C₁₉H2, C₂₂H2); 1.38-1.63 (m, 5H, C₁₇H2, C₂₄H2, C₂₂H2); 2.00 (q, 2H, C₂H2); 2.2 (m, 2H, C₆H2); 2.25, 2.48 (m, 2H, C₁₃H2); 2.58-2.8 (m, 4H, C₃₇H2, C₂₅H2); 2.81 (m, 3H, C₂₁H3); 3.0, 3.3 (m, 2H, C₂₇H2); 3.22 (m, 2H, C₁₆H2); 3.92 (m, 1H, C₁₂H); 4.3 (m, 1H, C₅H); 4.52, 4.72 (m, 2H, C₄₄H2); 4.92 (m, 1H, C₉H); 6.96 (m, 1H, C₃₄H); 6.98 (m, 3H, C₂₉H, C₄₁H, C₄₈H); 7.05 (m, 1H, C₃₃H); 7.11 (m, 4H, C₃₉H, C₄₃H, C₄₆H, C₅₀H); 7.20 (m, 4H, C₄₀H, C₄₂H, C₄₇H, C₄₉H); 7.35 (m, 1H, C₃₂H); 7.4 (br s, 1H, N₄H); 7.49 (d, 1H, C₃₅H); 7.6 (br s, 3H, N₂₆H3⁺); 10.4 (s, 1H, N₃₀H).

IR (microscope in transmission, cm-1): v=3282 (NH), 3062, 2934 (CH), 2871, 1673, 1647 (4× C═O), 1545 (amid), 1496, 1455, 1355, 1287, 1202, 1177, 1133, 1030, 919, 833, 799, 743, 721, 701.

### Propionyl-(S)-β³-HPhe-(R)—N-Benzyl-Trp-(S)-β³-HLys-methyl-hexyl-amide (Compound 69)

22 mg, >99% purity, Rt=8.40, MS [M+H]⁺ 751.9, HRMS [M+H]⁺ 751.4903 (calcd 751.4905), [M+Na]⁺ 773.4724 (calcd 773.4725).

### Propionyl-(S)-β³-HPhe-(R)—N-Me-Trp-(S)-β³-HLys-pentyl-amide (Compound 70)

70 mg, >95% purity, Rt=7.14, MS [M+H]⁺ 47.2, [M+2H]²⁺ 324.7, [M−H]⁻ 645.3, [M+TFA]⁻ 759.2, HRMS [M+H]⁺ 647.4277 (calcd 647.4279).

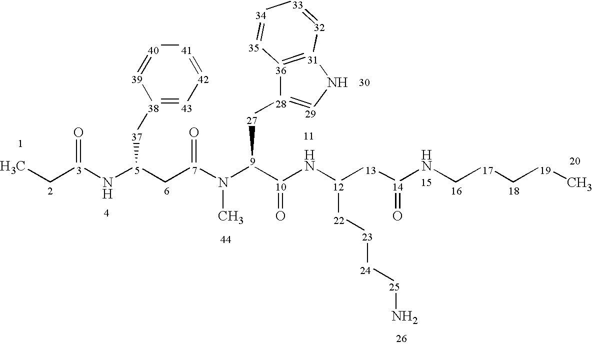

¹H (500 MHz, DMSO, mixture of rotamers; T=393K): δ (ppm)=0.86 (m, 3H, C₂₀H3); 0.92 (m, 3H, C₁H3); 1.18-1.33 (m, 6H, C₁₈H2, C₂₃H2, C₁₉H2); 1.37-1.53 (m, 6H, C₁₇H2, C₂₄H2, C₂₂H2); 1.98 (q, 2H, C₂H2); 2.28 (m, 2H, C₆H2); 2.28, 2.4 (m, 2H, C₁₃H2); 2.62 (m, 2H, C₂₅H2); 2.68-2.8 (m, 2H, C₃₇H2); 2.86 (m, 3H, C₄₄H3); 3.0, 3.3 (m, 2H, C₂₇H2); 3.05 (m, 2H, C₁₆H2); 4.05 (m, 1H, C₁₂H); 4.22 (m, 1H, C₅H); 5.15 (m, 1H, C₉H); 6.97 (m, 1H, C₃₄H); 7.02 (m, 2H, C₃₃H, C₂₉H); 7.09 (m, 2H, C₃₉H, C₄₃H); 7.12 (m, C₄₁H); 7.18 (m, 2H, C₄₀H, C₄₂H); 7.30 (m, 1H, C₃₂H); 7.52 (d, 1H, C₃₅H); 10.38 (s, 1H, N₃₀H).

IR (microscope in transmission, cm⁻¹): v=3286 (NH), 3062, 2934 (CH), 1648 (4× C═O), 1546 (amid), 1456, 1356, 1291, 1202, 1178, 1133, 1031, 1011, 919, 834, 799, 744, 721, 701.

### (S)-β³-HPhe-(R)—N-Me-Trp-(S)-β³-HLys-NH₂ (Compound 72)

30 mg, >99% purity, Rt=5.28, MS [M+H]⁺ 521.8, [M+2H]²⁺ 261.6, HRMS [M+H]⁺ 521.3236 (calcd 521.3235), [M+Na]⁺ 543.3057 (calcd 543.3054).

An overview of the physicochemical properties of the named compounds is given in Table 3.

TABLE 3

| | | | | | | | HT | | log P/D |
|---|---|---|---|---|---|---|---|---|---|
| Compound | HT-Solubility | HT-Solubility | HT-Permeability | HT-Permeability | HT-Permeability | Permeability | LogP | | logP |
| Number | pH 6.8 \|g 1-1\| | pH 1.0 \|g 1-1\| | logPe pH 4.0 | logPe pH 6.8 | logPe pH 8.0 | Class | ohw | CLO-GP | octanol |
| 1 | | | | | | | | | |
| 2 | 0.0133 | 0.0133 | −7 | −7 | −7 | L | | 1.1 | |
| 3 | 0.0133 | 0.0133 | −7 | −7 | −7 | L | | 1.1 | 0.4 |
| 4 | | | | | | | | | |
| 5 | 0.0143 | 0.0357 | −7 | −7 | −7 | L | 3.9 | 4 | |
| 0 | 0.0142 | 0.1418 | −7 | −7 | −7 | L | 2.8 | 3.7 | |
| 7 | 0.1294 | 0.1294 | −4.9 | −4.8 | −4.8 | M | | 2 | |
| 8 | 0.1294 | 0.1294 | −6.1 | −5.5 | −5.7 | L | 3.4 | 2 | 0.4 |
| 9 | | | | | | | | | |
| 10 | 0.0074 | 0.0368 | −7 | −7 | −7 | L | 2.7 | 4.1 | |
| 11 | 0.0149 | 0.0149 | −7 | −7 | −7 | L | 3.9 | 5.7 | |
| 12 | | | | | | | | | |
| 13 | 0.0767 | 0.1534 | −5.5 | 6.2 | −5 | L | 2.8 | 4.6 | |
| 14 | 0.0145 | 0.1452 | −7 | −7 | −7 | L | 2.6 | 2.8 | |
| 15 | 0.0151 | 0.0377 | −5.4 | −5.2 | −4.9 | L | 2.7 | 3.2 | |
| 16 | | | | | | | | | |
| 17 | 0.0152 | 0.0152 | −4.9 | −5.6 | 4.8 | M | 4.2 | 4.8 | |
| 18 | 0.0157 | 0.0784 | −7 | −7 | −7 | L | 3.2 | 3.8 | |
| 19 | 0.015 | 0.015 | −7 | −7 | −7 | L | 3.7 | 4.5 | |
| 20 | 0.0156 | 0.0156 | −7 | −7 | −7 | L | 2.9 | 5.1 | |
| 21 | 0.0132 | 0.0132 | −7 | −7 | −7 | L | 3.3 | 2.4 | |
| 22 | 0.0144 | 0.0144 | −7 | −7 | −7 | L | 2.3 | 3.6 | |
| 23 | 0.0174 | 0.0174 | −7 | −7 | −7 | L | 5.9 | 6.7 | |
| 24 | 0.0162 | 0.0162 | −7 | −7 | −7 | L | 4.1 | 5.7 | |
| 25 | 0.0156 | 0.078 | −7 | −7 | −7 | L | 3.3 | 4.6 | |
| 26 | 0.0015 | 0.0073 | −7 | −7 | −7 | L | 3.3 | 3.2 | |
| 27 | 0.009 | 0.045 | | | | | | | |
| 28 | 0.0605 | 0.0605 | −6 | −5.9 | −5.5 | L | 3.5 | 2.1 | |
| 29 | 0.121 | 0.121 | −5.4 | −5.5 | −5.4 | L | 3.3 | 2.1 | |
| 30 | | | | | | | | | |
| 31 | 0.1234 | 0.1234 | −5.6 | −5.7 | −6 | L | 2.8 | 2.7 | |
| 32 | 0.0141 | 0.0707 | −7 | −7 | −7 | L | 4 | 4.8 | |
| 33 | | | | | | | | | |
| 34 | 0.0737 | 0.1474 | −5.3 | −5.2 | −5.9 | L | 5.2 | 5.4 | |
| 35 | | | | | | | | | |
| 36 | 0.1244 | 0.1244 | −5.8 | −5.8 | −5.7 | L | | 1.2 | |
| 37 | 0.0675 | 0.135 | −5.2 | −5.3 | −5.3 | L | 2.4 | 3 | |
| 38 | 0.0309 | 0.1238 | −7 | −7 | −7 | L | 3.2 | 2.4 | |
| 39 | 0.1322 | 0.1322 | −5.3 | −5.7 | −5.5 | L | 3.4 | 2.4 | |
| 40 | 0.0132 | 0.0661 | −7 | −7 | −7 | L | 3.3 | 2.5 | |
| 41 | 0.1322 | 0.1322 | −5.6 | −5.4 | −6 | L | 3.6 | 4.8 | |
| 42 | 0.1294 | 0.0647 | −6 | −5.7 | −5.3 | L | 3.7 | 4.3 | |
| 43 | 0.1265 | 0.1266 | −6 | −5.7 | −5.9 | L | 3 | 3.8 | |
| 44 | 0.0309 | 0.1236 | −5.2 | −4.9 | −4.6 | M | 3.3 | 3.3 | |
| 45 | 0.0619 | 0.1236 | −5.4 | −5.5 | −5.5 | L | 3.2 | 3.3 | |
| 46 | 0.129 | 0.129 | −6 | −6 | −6 | L | 3.3 | 3.6 | |
| 47 | 0.1266 | 0.1266 | −5.4 | −5.8 | −6 | L | 3 | 3.8 | |
| 48 | 0.1294 | 0.1294 | −5.5 | −5.8 | −5.4 | L | 3.4 | 4.3 | |
| 49 | 0.1294 | 0.1294 | −5.3 | −6 | −5.7 | L | 3.7 | 4.3 | 2.8 |
| 50 | 0.0127 | 0.1266 | −7 | −7 | −7 | L | 3.4 | 3.9 | |
| 51 | 0.0316 | 0.1266 | −5.1 | −5.1 | −5.4 | L | 3.4 | 3.9 | |
| 52 | 0.121 | 0.121 | −5.2 | −5.8 | −6 | L | 3.1 | 2.8 | |
| 53 | 0.0309 | 0.1238 | −5.1 | −5.3 | −5 | L | 3.1 | 3.3 | |
| 54 | 0.0127 | 0.1266 | −7 | −7 | −7 | L | 3.5 | 3.9 | |
| 55 | 0.0129 | 0.0647 | −5.6 | −7 | −7 | L | 3.7 | 4.4 | |
| 56 | 0.0129 | 0.129 | −7 | −7 | −7 | L | 3 | 3.6 | |
| 57 | 0.0631 | 0.1262 | −6 | −6 | −6 | L | 2.8 | 3.2 | |
| 58 | 0.1097 | 0.1097 | −6 | −5.3 | −5.3 | L | | 1 | |
| 59 | 0.0507 | 0.1013 | −6 | −5.4 | −5.5 | L | | 1.1 | 1 |
| 60 | 0.1125 | 0.1125 | −5.5 | −5.1 | −5.9 | L | | 1.5 | |
| 61 | 0.1154 | 0.1154 | −5.9 | −5.9 | −6 | L | 2.8 | 2.1 | |
| 62 | 0.115 | 0.115 | −5.3 | −5.3 | −5.4 | L | 2.9 | 2.8 | |
| 63 | 0.1322 | 0.1322 | −6 | −6 | −6 | L | 3.9 | 4.8 | |
| 64 | 0.1322 | 0.1322 | −5.6 | −5.2 | −5.7 | L | 3.7 | 4.8 | 3.1 |
| 65 | 0.135 | 0.135 | −4.9 | −5.6 | −5.3 | L | 4.1 | 5.4 | |
| 66 | 0.0147 | 0.1474 | −4.9 | −5.1 | −5.4 | L | 5.5 | 6.5 | |
| 67 | 0.0147 | 0.1474 | −7 | −7 | −7 | L | 4.4 | 6.5 | |
| 68 | 0.015 | 0.0751 | −7 | −7 | −7 | L | 5.5 | 7 | |
| 69 | 0.015 | 0.0751 | −7 | −7 | −7 | L | 5.1 | 7 | |
| 70 | 0.1294 | 0.1294 | −5.3 | −5.4 | −4.9 | L | 3.3 | 4.4 | |
| 71 | 0.026 | 0.026 | | | | | | 1.7 | |
| 72 | 0.1041 | 0.1041 | −5.3 | −5.1 | −5.2 | L | | 1.7 | 1.5 |

### REFERENCES

Abraham, M, et al (1999) The Correlation and Prediction of the Solubility of Compounds in Water Using an Amended Solvation Energy Relationship. J. Pharm. Sci.; 88 (9): 868-880.
Box, K, et al (2003) High-Throughput Measurements of pKa values in a mixed Linear pH Gradient System. Anal. Chem.; 75: 883-892.
Corner, J E (2003) High-throughput Measurement of log D and pKₐ in Drug Bioavailability; Chapter 2: 21-45.
Faller, B, et al (2004) High-Throughput Lipophilicity Measurement with Immobilized Artificial Membranes (OCT-PAMPA). J. Med. Chem.; submitted for publication.
Faller, B (2003) Strategies to Improve Solubility. Congress presentation, Brussels (October 2-3).
Gademann, K, et al (2000) The Cyclo-β-Tetrapeptide (β-HPhe-β-HThr-β-HLys-β-HTrp): Synthesis, NMR Structure in Methanol Solution and Affinity for Human Somatostatin Receptors. Helvetica Chimica Acta; 83: 16-33.
Gademann, K, et al (2001) Peptide Folding Induces High and Selective Affinity of a Linear and small β-Peptide to the Human Somatostatin Receptor 4. J. Med. Chem.; 44 (15): 2460-2468.
Grace, Rani R G, et al (2003) Novel sst₄-Selective Somatostatin (SRIF) Agonists. 4. Three-Dimensional Consensus Structure by NMR. J. Med. Chem.;
46 (26): 5606-5618.
Hirschmann, R, et al (2003) Effects of Heterocyclic Aromatic Substituents on Binding Affinities at Two Distinct Sites of Somatostatin Receptors. Correlation with the Electrostatic Potential of the Substituents. J. Med. Chem.; 46 (10): 1858-1869.
Levy, D, et al (1998) Matrix Metalloproteinase Inhibitors: A Structure-Activity Study. J. Med. Chem.; 41 (2): 199-223.
Lipinski, C A, et al (1997) Experimental and computational approaches to estimate solubility and permeability in drug discovery and development settings. Advanced Drug Delivery Reviews; 23: 3-25.
Liu, S. et al (1998) Nonpeptide Somatostatin agonists with sst4 selectivity: synthesis and structure-activity relationships of thioureas. J. Med. Chem.; 41 (24): 4693-4705.
Mentlein, R, et al (2001) Somatostatin inhibits the production of vascular endothelial growth factor in human glioma cells. Int. J. Cancer; 92 (4): 545-550.
Moneta, D, et al (2002) Somatostatin receptor subtypes 2 and 4 affect seizure susceptibility and hippocampal excitatory neurotransmission in mice. Eur. J. Neurosci.; 16 (5): 843-849.
Patel, Y. C (1999) Somatostatin and its receptor family. Front Neuroendocrinol. 20 (3): 157-198.
Quitt, P, et al (1963) Synthesis of optically active N-monomethyl-amino acids. Helvetica Chimica Acta; 46: 327-333.
Rivier, J, et al (2003) Novel sst4-Selective Somatostatin (SRIF) Agonists. 1. Lead Identification Using a Betide Scan. J. Med. Chem.; 46: 5579-5586.
Rohrer, S, et al (1998) Rapid Identification of Subtype-Selective Agonists of the Somatostatin Receptor Through Combinatorial Chemistry. Science; 282: 737-740.
Seebach, D, et al (2001) Linear, Peptidase-Resistant β²/β³-Di- and α/β³-Tetrapeptide Derivatives with Nanomolar Affinities to a Human Somatostatin Receptor. Helvetica Chimica Acta; 84: 3503-3510.
Souers, A, et al (2000) Optimization of a Somatostatin Mimetic via Constrained Amino Acid and Backbone Incorporation. Bioorg. Med. Chem. Lett.; 10, 2731-2733.
Weckbecker, G, et al (2003) Opportunities in somatostatin research: Biological, chemical and therapeutic aspects. Nature Reviews Drug Discovery; 2: 999-1017.
Wohnsland, F, et al (2001) High-Throughput Permeability pH Profile and High-Throughput Alkane/Water log P with Artificial Membranes. J. Med. Chem.; 44: 923-930.

1. A compound of Formula 1: 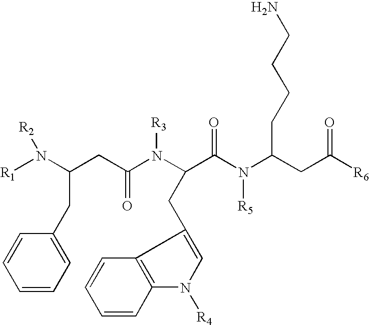
wherein R¹=COR⁷ or R⁷, wherein R⁷ is
a linear or branched C₁-C₁₂ alkyl group,
a linear or branched C₂-C₁₂ alkenyl group,
a linear or branched C₂-C₁₂ alkynyl group,
or a saturated/unsaturated, aromatic or heteroaromatic mono- or polycyclic group,
wherein said alkyl, alkenyl or alkynyl group may be mono- or polysubstituted with halo, hydroxy, C₁-C₄ alkoxy, carboxy, C₁-C₄ alkoxy carbonyl, amino, C₁-C₄ alkyl amino, di-(C₁-C₄-alkyl) amino, cyano, carboxy amide, carboxy-(C₁-C₄-alkyl) amino, carboxy-di(C₁-C₄-alkyl) amino, sulfo, sulfido (C₁-C₄-alkyl), sulfoxido (C₁-C₄-alkyl), sulfono (C₁-C₄-alkyl), thio or a saturated, unsaturated, aromatic or heteroaromatic, mono- or polycyclic group,
wherein said cyclic group may be mono- or polysubstituted with halo, hydroxy, C₁-C₄-alkoxy, carboxy C₁-C₄-alkoxycarbonyl, amino, C₁-C₄-alkylamino, di(C₁-C₄-alkyl) amino, cyano, carboxy amide, carboxy (C₁-C₄-alkyl) amido, carboxy-di(C₁-C₄-alkyl) amido, sulfo, sulfido (C₁-C₄-alkyl), sulfoxido (C₁-C₄-alkyl), sulfono (C₁-C₄-alkyl), thio, C₁-C₄-alkyl, C₂-C₄ alkenyl or C₂-C₄ alkynyl;
R² is hydrogen or C₁-C₄ alkyl,
R³ is hydrogen or C₁-C₄ alkyl, which may be substituted with a saturated, unsaturated, aromatic or heteroaromatic, mono- or polycyclic group,
R⁴ is hydrogen or C₁-C₄ alkyl,
R⁵ is hydrogen or C₁-C₄ alkyl, and
R⁶=(Y)ₙ(—NR⁸R⁹)ₘ, wherein Y is the residue of an amino carboxylic acid, particularly of a β-aminocarboxyclic acid, wherein Y may form a cyclic group;
n=0 or 1,
m=0 or 1,
R⁸ and R⁹ are independently hydrogen,
a linear or branched C₁-C₁₂ alkyl group,
a linear or branched C₂-C₁₂ alkenyl group,
a linear or branched C₂-C₁₂ alkenyl group,
or a saturated, unsaturated, aromatic or heteroaromatic mono- or polycyclic group,
wherein said alkyl, alkenyl or alkynyl group may be mono- or polysubstituted with halo, hydroxy, C₁-C₄ alkoxy, carboxy, C₁-C₄ alkoxy carbonyl, amino, C₁-C₄ alkyl amino, di-(C₁-C₄-alkyl) amino, cyano, carboxy amide, carboxy-(C₁-C₄-alkyl) amino, carboxy-di(C₁-C₄-alkyl) amino, sulfo, sulfido (C₁-C₄-alkyl), sulfoxido (C₁-C₄-alkyl), sulfono (C₁-C₄-alkyl), thio or a saturated, unsaturated, aromatic or heteroaromatic, mono- or polycyclic group,
wherein said cyclic group may be mono- or polysubstituted with halo, hydroxy, C₁-C₄-alkoxy, carboxy C₁-C₄ alkoxycarbonyl, amino, C₁-C₄-alkylamino, di(C₁-C₄-alkyl) amino, cyano, carboxy amide, carboxy (C₁-C₄-alkyl) amido, carboxy-di(-C₁-C₄ alkyl) amido, sulfo, sulfido (C₁-C₄-alkyl), sulfoxido (C₁-C₄-alkyl), sulfono (C₁-C₄-alkyl), thio, C₁-C₄ alkyl, C₂-C₄ alkenyl or C₂-C₄ alkynyl;
or wherein R⁸ and R⁹ together form a cyclic group, preferably a 5- or 6-membered cyclic group;
or a salt or derivative thereof in the form of an individual enantiomer, diastereomer or a mixture thereof.

2. The compound according to claim 1, wherein R⁷ is an optionally substituted C₁-C₁₀ alkyl or an optionally substituted cyclic group.

3. The compound of claim 2, wherein R⁷ is methyl, ethyl, butyl, nonyl, phenyl, ethylphenyl, cyclohexyl or adamantyl.

4. The compound according to claim 1, wherein R² is hydrogen or methyl.

5. The compound according to claim 1, wherein R³ is hydrogen, methyl or ethylphenyl.

6. The compound according to claim 1, wherein R⁴ is hydrogen or methyl.

7. The compound according to claim 1, wherein R⁵ is hydrogen or methyl.

8. The compound according to claim 1, wherein n=0.

9. The compound according to claim 1, wherein n=1.

10. The compound according to claim 9, wherein R⁶ is a β-threonine residue which may form a lactone group or a β-threonine amide residue.

11. The compound according to claim 9, wherein R⁶ is a β-valine residue or a β-valine amide residue.

12. The compound according to claim 1, wherein R⁸ is an optionally substituted C₁-C₁₀, particularly C₂-C₈ alkyl group or an optionally substituted cyclic group.

13. The compound according to claim 12, wherein R⁸ is ethyl, butyl, pentyl, hexyl, ethylphenyl or cyclopentyl.

14. The compound according to claim 1, wherein R⁹ is hydrogen or C₁-C₂ alkyl.

15. The compound of claim 1 which binds to a somatostatin receptor.

16. The compound of claim 15 which binds selectively to the somatostatin receptor sst4.

17. The compound of claim 16 which has a K_{D} affinity of ≦50 nM for the sst4 receptor.

18. A pharmaceutical composition comprising the compound of claim 1 or a physiologically acceptable salt or derivative thereof as an active agent.

19. The composition of claim 18 for therapeutic use.

20. The composition of claim 18 for diagnostic use.

21. A method for preventing or treating a disorder associated with somatostatin receptor sst4 dysfunction, comprising administering a pharmaceutically effective amount of a compound of claim 1 to a subject in need thereof.

22. The method of claim 21, wherein the subject is a mammal.

23. The method of claim 22, wherein the subject is a human.

24. A use of a compound of claim 1 in the preparation of a pharmaceutical composition for the treatment of a disorder of the central nervous system, in particular epilepsy, impaired behaviour, impaired learning and memory, attention deficit disorder and pain, neurological disorder, such as neurodegenerative diseases, in particular Alzheimer's disease, Parkinson's disease and multiple sclerosis.

25. A use of a compound of claim 1 in the preparation of a pharmaceutical composition for the treatment of hyperproliferative disorders, in particular of endocrine and solid tumors, for example for the treatment of acromegaly, melanomas, breast cancer, prostate adenomas and prostate cancer, lung cancer, bowel cancer, skin cancer and leukemias.

26. A use of a compound of claim 1 in the preparation of a pharmaceutical composition for the treatment of diseases associated with vascular remodelling such as restenosis or the treatment of chronic transplant rejection.

27. A use of a compound of claim 1 in the preparation of a pharmaceutical composition for the treatment of post-surgical symptoms, such as brain aneurysms and postsurgical vascular re-stenosis,

28. A use of a compound of claim 1 in the preparation of a pharmaceutical composition for the treatment of gastrointestinal disorders such as diarrhoea and chemotherapy-induced and AIDS-related diarrhoea, as well as in the treatment of acute variceal bleeding.

29. A use of a compound of claim 1 in the preparation of a pharmaceutical composition for the treatment of inflammatory disorders including inflammations of the joints, including arthritis and rheumatoid arthritis, and other arthritic disorders such as rheumatoid spondylitis.

30. A use of a compound of claim 1 in the preparation of a pharmaceutical composition for the treatment of psoriasis, atopic dermatitis, asthma.

31. A use of a compound of claim 1 in the preparation of a pharmaceutical composition for the treatment of Graves disease, inflammatory bowel disease, diabetic retinopathy, nephropathy, diabetic angiopathies and ischemic diseases, benign prostatic hyperplasia.

32. A use of a compound of claim 1 in the preparation of a pharmaceutical composition for the treatment of ocular diseases, for example, age-related macula degeneration and glaucoma diabetic retinopathy.


SEQUENCE LISTING (3 sequences)

SEQ ID NO: 1 - protein, 4 aa, Artificial Sequence
  Description of Artificial Sequence Synthetic peptide
FWKT

SEQ ID NO: 2 - protein, 4 aa, Artificial Sequence
  Description of Artificial Sequence Synthetic peptide
FWKL

SEQ ID NO: 3 - protein, 4 aa, Artificial Sequence
  Description of Artificial Sequence Synthetic peptide
FTKW